(19) United States
(12) Patent Application Publication
(10) Pub. No.: US 2026/0042736 A1
(43) Pub. Date: Feb. 12, 2026

(54) COMPOSITIONS AND METHODS FOR MAKING SCYTONEMIN AND ITS ANALOGS AND DERIVATIVES

(71) Applicant: Board of Regents of Nevada System of Higher Education on behalf of the University of Nevada Reno, Reno, NV (US)

(72) Inventors: Christopher S. Jeffrey, Reno, NV (US); Christopher J. Barile, Reno, NV (US)

(73) Assignee: Board of Regents of Nevada System of Higher Education on behalf of the University of Nevada Reno, Reno, NV (US)

(21) Appl. No.: 19/292,763

(22) Filed: Aug. 6, 2025

Related U.S. Application Data
(60) Provisional application No. 63/681,552, filed on Aug. 9, 2024.

(51) Int. Cl. C07D 209/94; C07D 487/18

(52) U.S. Cl. CPC C07D 209/94; C07D 487/18

(57) ABSTRACT

Disclosed herein are compositions and methods for making scytonemin, including ita precursors, analogs, and derivatives. In one specific embodiment, the method includes: converting a tryptophan-based starting material to an alkene monomer intermediate having a structure according to Formula I,

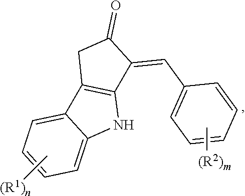

dimerizing the alkene monomer intermediate by exposing the alkene monomer intermediate to dimerization conditions, where dimerizing the alkene monomer intermediate produces the scytonemin analog; or dimerizing the alkene monomer intermediate produces a scytonemin compound precursor having a structure according to Formula II,

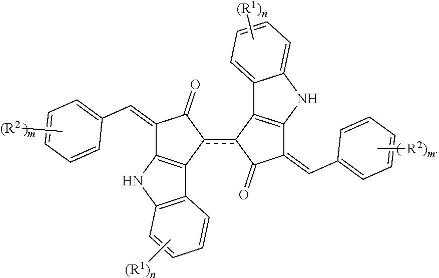

### CROSS-REFERENCE TO RELATED APPLICATION

This application claims the benefit of U.S. Provisional Patent Application No. 63/681,552, filed Aug. 9, 2024, which is incorporated by reference herein in its entirety.

### ACKNOWLEDGMENT OF GOVERNMENT SUPPORT

This invention was made with government support under Contract No. 80NSSC22K1633 awarded by National Aeronautics and Space Administration. The government has certain rights in the invention.

### BACKGROUND

### Field

Methods for making scytonemin, its precursors, and its derivates and analogs are described.

### Description of Related Art

Extracellular UV-absorbing microbial pigments play a crucial role in shielding organisms from environmental stresses caused by intense light, desiccation, and oxidative damage. One such pigment, scytonemin, found in the extracellular polysaccharide sheath of cyanobacteria, is believed to have originated over 2.1 billion years ago in some of the earliest cyanobacterial species. This compound is implicated as one of the key components that is proposed to have permitted life on early Earth, prior to the formation of the mature ozone layer. Scytonemin and other pigments have been proposed as cellular protectants as they function as extremely strong antioxidants. Scytonemin is produced by cyanobacteria and localized to the extracellular polysaccharide sheath and absorbs across the UV spectrum without blocking photosynthetically active light. Although there is debate about whether scytonemin or its biosynthetic precursors directly screened and dissipated lethal UV-C radiation, enabling cyanobacteria to survive before the ozone layer formed, it is undeniable that its broad-spectrum UV-absorbing and potential antioxidant properties would have aided organisms coping with intense UV radiation in an increasingly oxygen-rich environment. The remarkable survivability of organisms producing scytonemin under intense UV exposure, its induction in response to UV exposure, its ability to absorb a wide range of UV radiation, and its recognition as an effective antioxidant have spurred extensive research. Yet, detailed studies elucidating the structural aspects underlying its protective properties remain scarce, particularly those focused on its unique antioxidant characteristics that may have been integral to the essential protection of these organisms to ROS. Since the early descriptions of scytonemin's structure and characteristics, researchers have noted the variable presence of its khaki green oxidized form [O]-scytonemin (10) and red reduced form [H]-scytonemin (1 h) through thin layer chromatography and HPLC analyses of cyanobacterial extracts. These forms were observed to interchange under mild redox conditions, such as reduction by ascorbic acid and oxidation when exposed to air.

Isolation of scytonemin from natural sources has only produced milligram quantities from kilograms of biological tissue. In particular, access to scytonemin from biologic material is strenuous and low yielding, producing only ca. 1 mg per Kg of dry tissue as a mixture of reduced and oxidized analogs. As such, this limits the extent of the ultrafast photophysical and other studies that can be conducted on the compound and clearly limits its supply for any potential uses as a therapeutic and/or protectant. Methods for making scytonemin exist in the art; however, these methods have associated drawbacks that have led to difficulties in producing meaningful amounts of scytonemin, which in turn leads to the inability to harness its full potential as a therapeutic and/or protectant. And, chemical synthesis of scytonemin faces many challenges. For instance, certain intermediates used to arrive at scytonemin using conventional syntheses are prohibitively expensive due to the need for hazardous methods used to produce them (e.g., methods/reagents that pose explosion risks) and/or expensive reagents (e.g., exotic metal catalysts).

Consequently, there is a need for new methods for making scytonemin that are scalable and exhibits improved yields and can also provide the ability to arrive at new scytonemin derivatives.

### SUMMARY

Provided herein are compositions and methods for making scytonemin, scytonemin analogs, and scytonemin derivatives. In one specific embodiment, the method includes: converting a tryptophan-based starting material to an alkene monomer intermediate having a structure according to Formula I; dimerizing the alkene monomer intermediate by exposing the alkene monomer intermediate to dimerization conditions, wherein (i) dimerizing the alkene monomer intermediate produces the scytonemin analog; or (ii) dimerizing the alkene monomer intermediate produces a scytonemin compound precursor having a structure according to Formula II and the method further including oxidizing the scytonemin compound precursor to provide scytonemin or the scytonemin analog; wherein Formulas I and II are as described herein and, wherein with respect to these formulas, each R¹ independently is selected from aliphatic, heteroaliphatic, haloaliphatic, haloheteroaliphatic, aromatic, or an organic functional group, or two R¹ groups join together to form a fused ring system with carbon atoms to which the two R¹ groups are bound; each R² independently is selected from aliphatic, heteroaliphatic, haloaliphatic, haloheteroaliphatic, aromatic, or an organic functional group, or two R² groups join together to form a fused ring system with carbon atoms to which the two R² groups are bound; n is an integer selected from 0 to 4; and m is an integer selected from 0 to 5.

In any or all of the above aspects, converting the tryptophan-based starting material to the alkene monomer intermediate includes: (i) coupling the tryptophan-based starting material with an anhydride reagent according to Formula III as described herein to provide a trihalo oxazolone (wherein, with respect to Formula III, each X independently is a halogen); (ii) exposing the trihalo oxazolone to a Lewis acid to provide a ketoamide intermediate according to Formula IV as described herein (wherein, with respect to Formula IV, each X independently is a halogen; R¹ is selected from aliphatic, heteroaliphatic, haloaliphatic, haloheteroaliphatic, aromatic, or an organic functional group; and n is an integer selected from 0 to 4); (iii) oxidizing the ketoamide intermediate to a diketone intermediate; (iv) performing a Grignard addition reaction to functionalize the diketone intermediate at a position bearing a ketone of the diketone intermediate to provide an alpha-hydroxy ketone-containing compound; and (v) dehydrating the alpha-hydroxy ketone-containing compound to provide the alkene monomer intermediate.

Also disclosed is a compound having a formula according to Formula IIA or Formula IIB as described herein, wherein each R¹ independently is selected from aliphatic, heteroaliphatic, haloaliphatic, haloheteroaliphatic, aromatic, or an organic functional group, or two R¹ groups join together to form a fused ring system with carbon atoms to which the two R¹ groups are bound; each R² independently is selected from aliphatic, heteroaliphatic, haloaliphatic, haloheteroaliphatic, aromatic, or an organic functional group, or two R² groups join together to form a fused ring system with carbon atoms to which the two R² groups are bound; n is an integer selected from 0 to 4, and m is an integer selected from 0 to 5; provided that the compound is not scytonemin or scytonemin imine.

### BRIEF DESCRIPTION OF THE DRAWINGS

For the purpose of promoting an understanding of the principles of the present disclosure, reference is now made to the embodiments illustrated in the drawings, which are described below. The embodiments disclosed herein are not intended to be exhaustive or limit the present disclosure to the precise form disclosed in the following detailed description. Rather, the embodiments are chosen and described so that others skilled in the art can utilize their teachings. Therefore, no limitation of the scope of the present disclosure is thereby intended.

FIG. 1 shows the structures of the reduced form (top) and oxidized form (bottom) of scytonemin.

FIG. 2A shows a Randles-Sevcik plot obtained from CVs of a glassy carbon electrode in an electrolyte containing 5 mM scytonemin and 50 mM NaClO₄ in CH₃CN. FIG. 2B shows a Randles-Sevcik plot obtained from CVs of a glassy carbon electrode in an electrolyte containing 5 mM scytonemin and 50 mM NaClO₄ in CH₃CN.

FIG. 3 is a graph of current density (mA cm⁻²) as a function of voltage (V vs. Ag wire) showing representative cyclic voltammograms of a glassy carbon electrode of 5 mM ferrocene and 50 mM NaClO₄ in CH₃CN at 500 mV s⁻¹.

FIG. 4A is a graph of current density (mA cm⁻²) as a function of voltage (V vs. NHE) showing cyclic voltammograms for scytonemin in 1.2 M Britton-Robinson Buffer solution at pH values of 2.5. FIG. 4B is a graph of current density (mA cm⁻²) as a function of voltage (V vs. NHE) showing cyclic voltammograms for scytonemin in 1.2 M Britton-Robinson Buffer solution at pH values of 4.0. FIG. 4C is a graph of current density (mA cm⁻²) as a function of voltage (V vs. NHE) showing cyclic voltammograms for scytonemin in 1.2 M Britton-Robinson Buffer solution at pH values of 5.5. FIG. 4D is a graph of current density (mA cm⁻²) as a function of voltage (V vs. NHE) showing cyclic voltammograms for scytonemin in 1.2 M Britton-Robinson Buffer solution at pH values of 7. FIG. 4E is a graph of current density (mA cm⁻²) as a function of voltage (V vs. NHE) showing cyclic voltammograms for scytonemin in 1.2 M Britton-Robinson Buffer solution at pH values of 8.5. FIG. 4F is a graph of current density (mA cm⁻²) as a function of voltage (V vs. NHE) showing cyclic voltammograms for scytonemin in 1.2 M Britton-Robinson Buffer solution at pH values of 10.

FIG. 5A is a graph of current density (mA cm⁻²) as a function of voltage (V vs. NHE) showing cyclic voltammograms for di-O-methyl scytonemin in 1.2 M Britton-Robinson Buffer solution at pH values of 2.5. FIG. 5B is a graph of current density (mA cm⁻²) as a function of voltage (V vs. NHE) showing cyclic voltammograms for di-O-methyl scytonemin in 1.2 M Britton-Robinson Buffer solution at pH values of 4.0. FIG. 5C is a graph of current density (mA cm⁻²) as a function of voltage (V vs. NHE) showing cyclic voltammograms for di-O-methyl scytonemin in 1.2 M Britton-Robinson Buffer solution at pH values of 5.5, FIG. 5D is a graph of current density (mA cm⁻²) as a function of voltage (V vs. NHE) showing cyclic voltammograms for di-O-methyl scytonemin in 1.2 M Britton-Robinson Buffer solution at pH values of 7. FIG. 5E is a graph of current density (mA cm⁻²) as a function of voltage (V vs. NHE) showing cyclic voltammograms for di-O-methyl scytonemin in 1.2 M Britton-Robinson Buffer solution at pH values of 8.5. FIG. 5F is a graph of current density (mA cm⁻²) as a function of voltage (V vs. NHE) showing cyclic voltammograms for di-O-methyl scytonemin in 1.2 M Britton-Robinson Buffer solution at pH values of 10.

FIG. 6A showS a ¹H nuclear magnetic resonance spectrum for a trihalo oxazolone intermediate compound described herein. FIG. 6B showS a ¹³C-nuclear magnetic resonance spectrum for a trihalo oxazolone intermediate compound described herein.

FIG. 7A shows a ¹H nuclear magnetic resonance spectrum for a ketoamide intermediate compound described herein. FIG. 7B shows a ¹³C-nuclear magnetic resonance spectrum for a ketoamide intermediate compound described herein.

FIG. 8A shows a H nuclear magnetic resonance spectrum of a diketone intermediate compound described herein. FIG. 8B shows a ¹³C-nuclear magnetic resonance spectrum for a diketone intermediate compound described herein.

FIG. 9A shows a ¹H nuclear magnetic resonance spectrum for an alkene monomer compound described herein. FIG. 9B shows a ¹³C-nuclear magnetic resonance spectrum for an alkene monomer compound described herein.

FIG. 10 is a graph of current density (mA cm⁻²) as a function of voltage (V vs. NHE) showing representative cyclic voltammograms of a glassy carbon electrode of 5 mM scytonemin and 50 mM NaClO₄ in CH₃CN at different scan rates.

FIGS. 11A-11C show the results for spectroelectrochemical analysis of 1 mM scytonemin and 50 mM NaClO₄ in DMSO using indium tin oxide on glass as a transparent conducting electrode. FIG. 11A shows the CV of scytonemin before and after 43 hours of chronoamperometry at +1.245 V vs. NHE at a scan rate of 10 mV s⁻¹. FIG. 11B shows UV-visible absorbance spectra of the solution at different times during the oxidation. FIG. 11C shows a chronoampermetric trace during oxidation.

FIGS. 12A-12B show results from analyzing scytonemin and di-O-methyl scytonemin immobilized on glassy carbon electrodes. FIG. 12A shows a CV scan taken at 1500 mV s⁻¹ in 1.2 M pH 8.5 Britton-Robinson buffer, with the inset magnifying the capacitance-subtracted portion of the di-O-methyl scytonemin CV that exhibits two reductive peaks. FIG. 12B shows the midpoint potential of scytonemin (□) and di-O-methyl scytonemin (⋅) versus pH with linear regression analysis (dashed lines).

FIG. 13 is a graph of disk current density (mA cm⁻²) as a function of voltage (V vs. NHE) showing rotating-ring disk electrochemistry of scytonemin at pH 7.0 (A).

FIG. 14A is a graph of current density (mA cm⁻²) as a function of voltage (V vs. NHE) showing representative cyclic voltammograms of a glassy carbon electrode of 5 mM Curcumin containing 50 mM NaClO₄ in CH₃CN at different scan rates. FIG. 14B is a graph of current density (mA cm⁻²) as a function of voltage (V vs. NHE) showing representative cyclic voltammograms of a glassy carbon electrode of 5 mM Daidzein containing 50 mM NaClO₄ in CH₃CN at different scan rates.

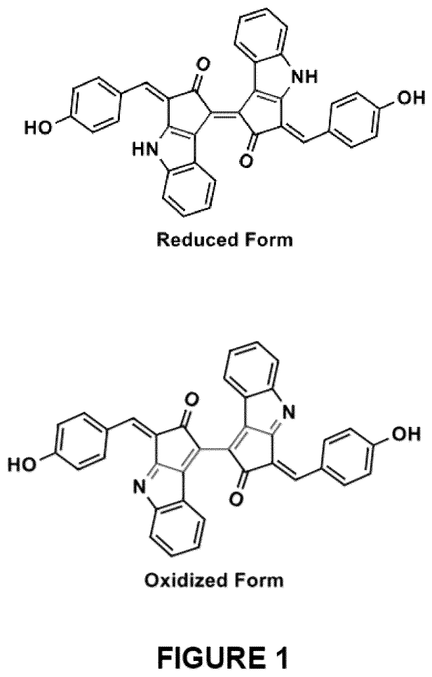

### DETAILED DESCRIPTION

In one or more embodiments, the method of making scytonemin, a scytonemin analog, or a scytonemin derivative includes converting a tryptophan-based starting material to an alkene monomer intermediate having a structure according to Formula I.

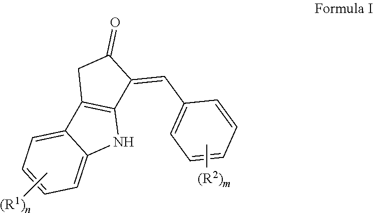

With reference to Formula I, each R¹ independently is selected from aliphatic, heteroaliphatic, haloaliphatic, haloheteroaliphatic, aromatic, or an organic functional group; each R² independently is selected from aliphatic, heteroaliphatic, haloaliphatic, haloheteroaliphatic, aromatic, or an organic functional group; n is an integer selected from 0 to 4, and m is an integer selected from 0 to 5. In some aspects of the disclosure, each R¹, each R², n, and m can be as described herein for any of Schemes 1, 1A, 2, or 2A as described herein.

The method can further include dimerizing the alkene monomer intermediate of Formula I to provide a scytonemin compound precursor having a structure according to Formula II by exposing the alkene monomer intermediate to dimerization conditions, wherein Formula II is

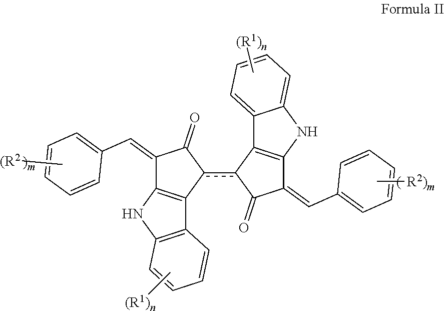

With reference to Formula II, each R¹ independently is selected from aliphatic, heteroaliphatic, haloaliphatic, haloheteroaliphatic, aromatic, or an organic functional group; each R² independently is selected from aliphatic, heteroaliphatic, haloaliphatic, haloheteroaliphatic, aromatic, or an organic functional group; n is an integer selected from 0 to 4, and m is an integer selected from 0 to 5. In some aspects of the disclosure, each R¹, each R², n, and m can be as described herein for any of Schemes 1, 1A, 2, or 2A as described herein.

The method can further include oxidizing the scytonemin compound precursor to provide scytonemin or an analog or derivative thereof. In some aspects, the method can further include a deprotection step wherein R² groups include any protecting group (e.g., hydroxyl, amine, and/or thiol protecting groups) are converted to deprotected forms of the R² group (e.g., converted to the corresponding hydroxyl, amine, and/or thiol groups).

To make the alkene monomer intermediate, the tryptophan-based starting material is coupled with an anhydride reagent according to Formula III to provide a trihalo oxazolone, wherein each X of Formula III independently is a halogen.

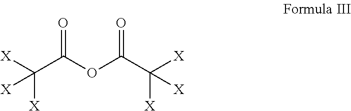

The method further includes exposing the trihalo oxazolone to a Lewis acid to provide a ketoamide intermediate according to Formula IV, wherein each X independently is a halogen; R¹ is selected from aliphatic, heteroaliphatic, haloaliphatic, haloheteroaliphatic, aromatic, or an organic functional group; and n is an integer selected from 0 to 4.

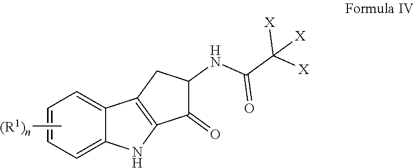

In aspects of the disclosure, the method further includes (i) oxidizing the ketoamide intermediate to a diketone intermediate; (iv) performing a Grignard addition reaction to functionalize the diketone intermediate at a position bearing a ketone of the diketone intermediate to provide an alpha-hydroxy ketone-containing compound; and (v) dehydrating the alpha-hydroxy ketone-containing compound to provide the alkene monomer intermediate.

In some aspects, the tryptophan-based starting material has a structure according to Formula V, wherein R¹ and n are as described herein for other formulas.

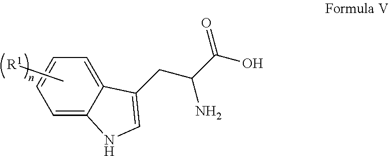

In some aspects, the diketone intermediate has a structure according to Formula VI wherein R¹ and n are as described herein for other formulas.

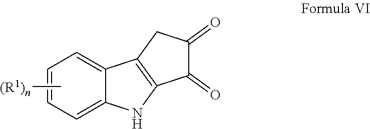

In some aspects, performing the Grignard addition reaction includes exposing the diketone intermediate to a reagent having a structure according to a Formula VII, wherein R² and m are as described herein for other formulas.

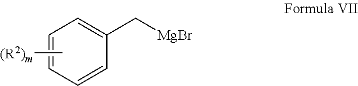

In some aspects, the alpha-hydroxy ketone-containing compound has a structure according to Formula VIII, wherein R¹, R², and n are as described herein for other formulas.

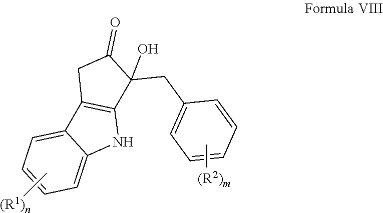

According to aspects of the present disclosure, precursors to scytonemin and analogs/derivatives of scytonemin can be made according to a method as illustrated in Scheme 1. With reference to Scheme 1, tryptophan-based starting material 100 can be reacted with anhydride reagent 102 to provide trihalo oxazolone 104. With reference to anhydride reagent 102, each X independently can be a halogen atom, such as Cl, F, I, or Br. In particular aspects, each X is selected from Cl, F, Br, or I. In exemplary aspects, each X is selected from Cl or F. Trihalo oxazolone 104 is exposed to a Lewis acid to promote an electrophilic aromatic substitution reaction and cyclization to ketoamide 106. In some aspects, the Lewis acid can be selected from AlCl₃, BF₃·OEt₂, FeCl₃, methanesulfonic acid, polyphosphoric acid, and the like. Oxidation and hydrolysis of ketoamide 106 to diketone 108 can be carried out using reagents known to those in the art with the benefit of the present disclosure, such as CuBr₂, Br₂, N-bromosuccinimide (“NBS”), I₂, PhI(OAc)₂, tBuOCl, and the like. In particular aspects of the disclosure, CuBr₂ is used. Treatment of diketone 108 with Grignard reagent 110 is carried out to provide alpha-hydroxy ketone intermediate 112, which then undergoes dehydration with suitable reagents to provide alkene monomer 114. In some aspects, the dehydration step is carried out using SOCl₂ or POCl₃ in the presence of a base, such as pyridine or the like. In some aspects, a Bronsted acid, can be used, such as para-toluene sulfonic acid, sulfuric acid, or the like.

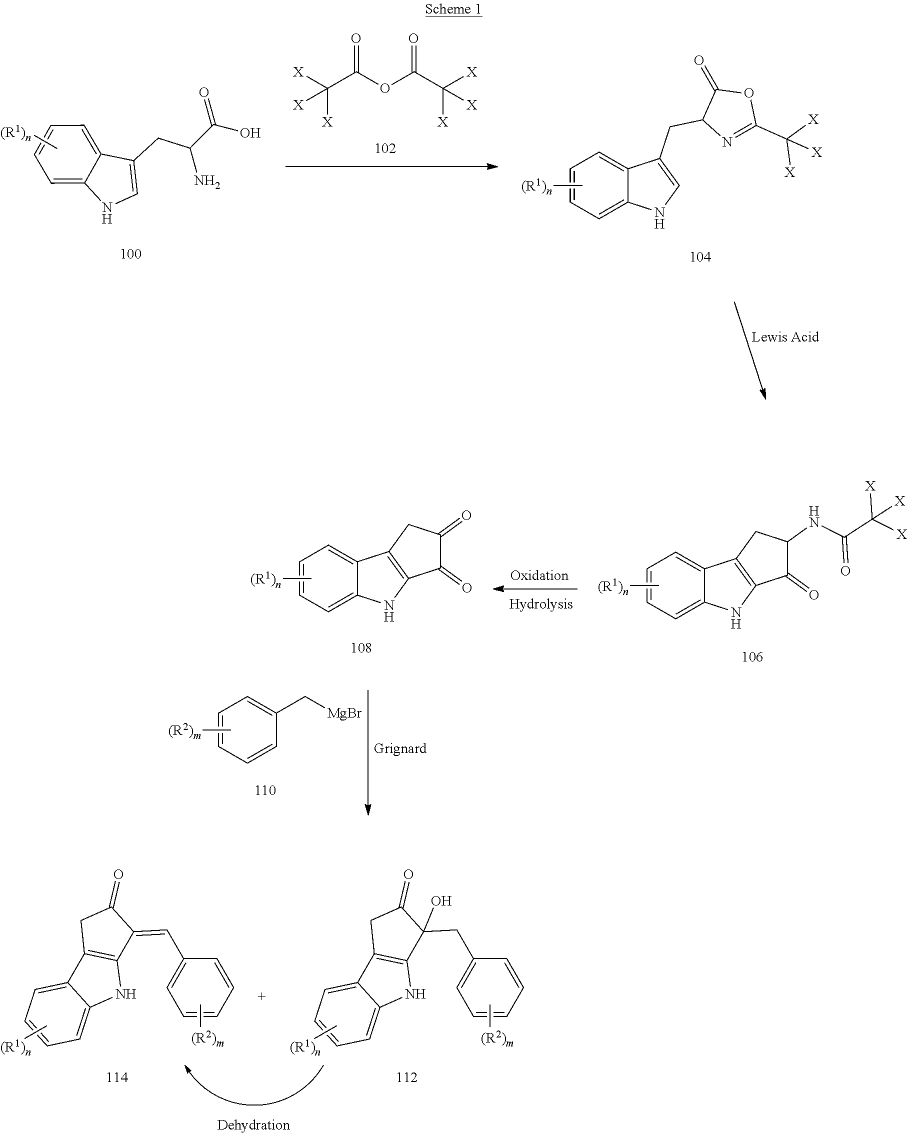

With reference to Scheme 1 and each of Formulas I and II discussed herein, each R¹ independently can be selected from aliphatic, heteroaliphatic, haloaliphatic, haloheteroaliphatic, aromatic, or an organic functional group; n can be an integer selected from 0 to 4, such as 0, 1, 2, 3, or 4; each R² independently can be selected from aliphatic, heteroaliphatic, haloaliphatic, haloheteroaliphatic, aromatic, or an organic functional group; and m can be an integer selected from 0 to 5, such as 0, 1, 2, 3, 4, or 5. In some aspects, two R¹ groups can be present and can join together to form a fused ring system with the aryl ring to which the two R¹ groups are attached. In some aspects, two R² groups can be present and can join together to form a fused ring system with the aryl ring to which the two R² groups are attached.

In some aspects of the disclosure, each R¹ independently can be selected from alkyl (e.g., lower alkyl, such as methyl, ethyl, propyl, butyl, pentyl, hexyl, heptyl, octyl, nonyl, or decyl), alkenyl, alkynyl, heteroalkyl (e.g., thioether, ether, methoxy, ethoxy, propoxy, butoxy, secondary amine, etc.), heteroalkenyl, heteroalkynyl, haloalkyl (e.g., CF₃ and the like), haloalkenyl, haloalkynyl, haloheteroalkyl (e.g., OCF₃ and the like), haloheteroalkenyl, haloheteroalkynyl, aryl, heteroaryl, or an organic functional group selected from OH, SH, NH₂, nitro, cyano, and the like. In some aspect, R¹ can be an ether group that is a polyalkylene oxide (e.g., a PEG group). In some aspects, each n is 1 and R¹ is positioned one, two, or three carbon atoms away from the phenyl carbon atom bearing the NH group of the illustrated indole. In exemplary aspects, each n is 0 and R¹ is not present. In some aspects, each R² independently can be selected from alkyl (e.g., lower alkyl, such as methyl, ethyl, propyl, butyl, pentyl, hexyl, heptyl, octyl, nonyl, or decyl), alkenyl, alkynyl, heteroalkyl (e.g., methoxy, ethoxy, propoxy, butoxy, and the like), heteroalkenyl, heteroalkynyl, haloalkyl (e.g., CF₃ and the like), haloalkenyl, haloalkynyl, haloheteroalkyl (e.g., OCF₃ and the like), haloheteroalkenyl, haloheteroalkynyl, aryl, heteroaryl, or an organic functional group selected from OH, SH, NH₂, nitro, cyano and the like. In some aspect, R¹ can be an ether group that is a polyalkylene oxide (e.g., a PEG group). In some aspects, each m is 1 and R² is positioned meta, ortho, or para relative to the bond connecting the aryl ring to which R² is attached to the rest of the compound. In exemplary aspects, each m is 1 and R² is positioned para relative to the bond connecting the aryl ring to which R² is attached to the rest of the compound. In such exemplary aspects, R² is selected from OMe, OCF₃, or t-butyl.

In some aspects, R¹, R², and R³ of the above formulas can be independently selected from the group consisting of: H; F; Cl; Br; I; OH; ketone (=O); ether [—OR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; acyl halide (—COX); carbonyl [—COR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]: aldehyde (—CHO); carbonate ester [—OCOOR, where R can include methyl, ethyl, n-propyl, isopropyl, n-butyl, sec-butyl, isobutyl, tert-butyl; (C₂₋₄) alkenyl, such as ethenyl, propenyl, butenyl, where the double bond can be located at any position in the alkenyl carbon chain, and including any alkenyl conformational isomers]; carboxyl (—COOH); amide [—CONR′R″, where R′ and R″ can be independently selected from hydrogen, methyl, ethyl, n-propyl, isopropyl, n-butyl, sec-butyl, isobutyl, tert-butyl; (C₂₋₄₋)alkenyl, such as ethenyl, propenyl, butenyl, where the double bond can be located at any position in the alkenyl carbon chain, and including any alkenyl conformational isomers]: amines [—NR′R″, where R′ and R″ can be independently selected from hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; cyanate (—OCN); isocynate (—NCO): nitrate (—ONO₂); nitrile (—CN); isonitrile (—NC); nitroso (—NO); oxime —CH═NOH); borono —B(OH)₂; boronare [—B(OR′)(R″), where R′ and R″ can be independently selected from hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; borinate [—B(R′)(OR″), where R′ and R″ can be independently selected from hydrogen, methyl, ethyl, n-propyl, isopropyl, n-butyl, sec-butyl, isobutyl, tert-butyl; (C₂)alkenyl, such as ethenyl, propenyl, butenyl, where the double bond can be located at any position in the alkenyl carbon chain, and including any alkenyl conformational isomers]; phosophino [—PR₂, where R can include hydrogen, methyl, ethyl, n-propyl, isopropyl, n-butyl, sec-butyl, isobutyl, tert-butyl; (C₂)alkenyl, such as ethenyl, propenyl, butenyl, where the double bond can be located at any position in the alkenyl carbon chain, and including any alkenyl conformational isomers]; phosphono [—P(═O)(OH)(R), where R can include hydrogen, methyl, ethyl, n-propyl, isopropyl, n-butyl, sec-butyl, isobutyl, tert-butyl: (C₂₋₄)alkenyl, such as ethenyl, propenyl, butenyl, where the double bond can be located at any position in the alkenyl carbon chain, and including any alkenyl conformational isomers]; phosphate (—OP(═O)(OH)₂; thiol (—SH); sulfide [—SR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; disulfide [—SSR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers], sulfinyl [—S(═O)R, where R can include methyl, ethyl, n-propyl, isopropyl, n-butyl, sec-butyl, isobutyl, tert-butyl: (C₂₋₄)alkenyl, such as ethenyl, propenyl, butenyl, where the double bond can be located at any position in the alkenyl carbon chain, and including any alkenyl conformational isomers]; sulfino (—SO₂H); sulfo (—SO₃H); thiocyanate; isothiocyanate; carbonothioyl [—C(═S)R where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; (C₁₋₄)alkyl, such as methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers; and where X can be independently selected from the group consisting of: CH, S, NH, and O.

Alkene monomer 114 from Scheme 1 can be converted to scytonemin or an analog or derivative thereof using the steps illustrated in Scheme 2, below. In particular aspects, alkene monomer 114 is converted to dimer 200 using reagents and/or reaction conditions that facilitate dimerizing alkene monomer 114. Such conditions for dimerization do not include using lithium diisopropylamide (or “LDA”). In particular aspects, the dimerization is carried out using suitable oxidants or by using electrochemical reaction conditions. In some aspects, suitable oxidants can include Cu(OAc)₂, AgF, PhI(OAc)₂, I₂, K₃[Fe(CN)₆], FeCl₃, and the like. In yet other aspects, electrochemical reaction conditions can be used and can include exposing the alkene monomer intermediate to a basic methanol solution in an electrochemical cell including a working electrode and a counter electrode operated at an operating potential. In particular aspects, the electrochemical conditions include oxidation of a solution of the monomer in basic methanol (e.g., a composition including NaOH, MeOH, and H₂O) in an electrochemical cell using a vitreous carbon working electrode and platinum wire counter electrode (e.g., Ag/AgCl reference) at the appropriate potential (e.g., 1.0 V). Upon dimerization, one or both of dimers 200 and 202 can be isolated. In some aspects, a mixture of dimers 200 and 202 is obtained. In other aspects, just dimer 200 or just dimer 202 is obtained. Each of these compounds can be converted to product 204 using an oxidant, such as 2,3-dichloro-5,6-dicyanobenzoquinone (or “DDQ”). In some aspects, dimer 202 can naturally oxidize to product 204 under ambient oxygen conditions. In some additional aspects, the dimerization step can also result in dimerization and direct oxidation to product 204 without isolating (or substantially producing) dimers 200 or 202 (illustrated in Scheme 2 with the dashed arrow from alkene monomer 114 to product 204). In some aspects where R² is an alkoxy group, dimerization conditions can be controlled to ensure that dimer 200 is obtained and, in such aspects, the dimer can be exposed to demethylation conditions (e.g., BBr₃) and then oxidized to product 204 (wherein R² is OH). In some aspects, deprotection can occur after product 204 is formed. For example, a further deprotection step can be used to convert one or more R² groups including a protecting group (e.g., hydroxyl-, amine-, and/or thiol-protecting groups) to deprotected forms of the R² group (e.g., converted to the corresponding hydroxyl, amine, and/or thiol groups).

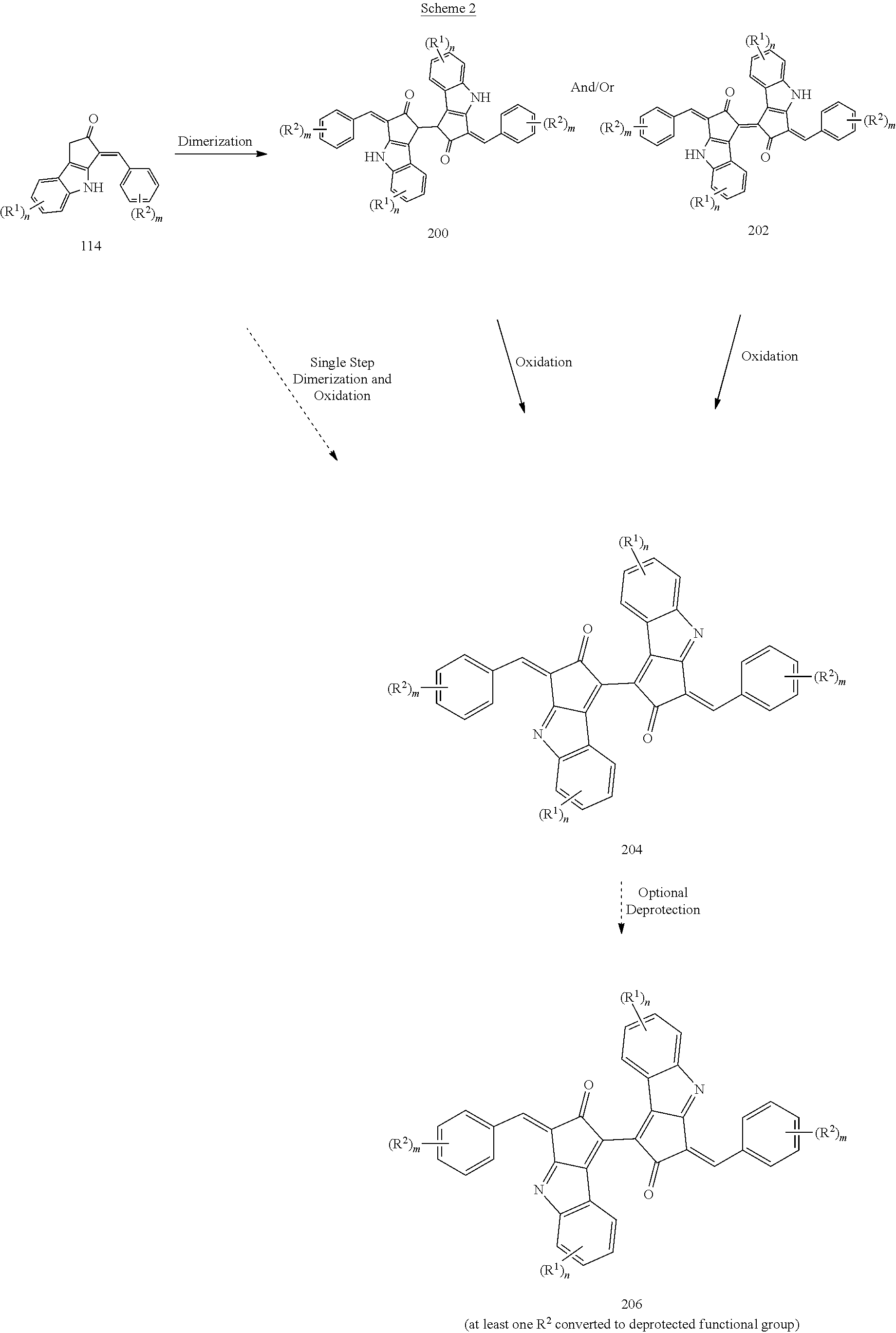

In some aspects of the disclosure, and with reference to Scheme 2 and each of Formulas I and II discussed herein, each R² can be a heteroaliphatic group, a haloheteroaliphatic group, or an organic functional group, wherein such groups include a protecting group component (e.g., an aliphatic, haloaliphatic group, or organic functional group that serves to protect a hydroxyl, thiol, or —NH₂ group). In some aspects, R² can be (i) a heteroaliphatic group selected from alkoxy groups, thioether groups, amine groups; (ii) a haloheteroaliphatic group like —OCF₃ and the like; or (iii) an organic functional group known in the art to be suitable for protecting a hydroxyl, thiol, or —NH₂ group (e.g., an alkoxy benzyl group, such as an ortho- or para-methoxy benzyl group, a dimethoxy benzyl group, or the like); wherein aliphatic, haloaliphatic, or organic functional group portions (e.g., an aliphatic group portion of the heteroaliphatic group, or a CF₃ group of a haloheteroaliphatic group, or an alkoxy benzyl group of an organic functional group) can be removed to thereby provide the deprotected hydroxyl, thiol, or —NH₂ group. In such aspects, the dimerization can take place with the heteroaliphatic, haloheteroaliphatic, or organic functional group present. As described above for Scheme 2, the optional deprotection step can be performed after oxidizing the dimerized compound; however, the deprotection step is optional and need not be performed in all aspects of the disclosure. In some aspects of the disclosure, the dimerization step can be performed using the electrochemical reaction conditions described herein, which can facilitate performing the dimerization with a free hydroxyl, thiol, or NH₂ group at the R² position and thus no protecting group is needed.

In some aspects, the method can further include exposing product 206 to an aqueous ammonia composition and a suitable carbonyl-containing reactant (e.g., a ketone or aldehyde reactant, such as, but not limited to, acetone, cyclohexanone, benzaldehyde, 4-t-butylcyclohexanone, and butyraldehyde) to form imine derivative 300, as shown below in Scheme 3. With reference to imine derivative 300, R³ can be aliphatic, aromatic, or can join with the carbon atom alpha to the imine carbon atom of Formula IIB to form a cyclic group. In some aspects, R³ is lower alkyl (e.g., methyl, ethyl, propyl, butyl, pentyl, hexyl, heptyl, octyl, nonyl, or decyl) or aryl (e.g., phenyl, naphthyl, or the like). In particular aspects, R³ is methyl, butyl, cyclohexyl, phenyl, 4-t-butylcyclohexyl, or forms a 6-membered ring with the carbon atom alpha to the imine carbon atom of compound 300. With reference to compound 300, any and all diastereomers are contemplated.

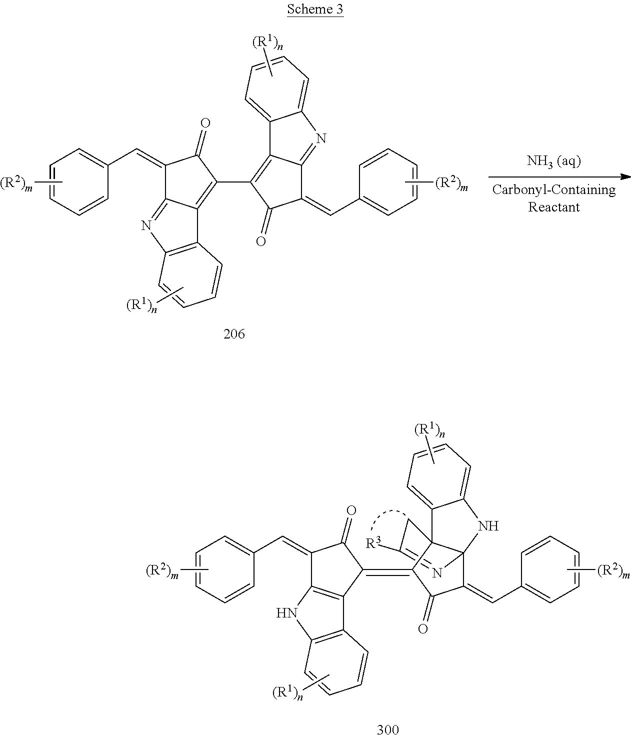

A representative method and representative conditions and reagents according to the present disclosure are shown in Schemes 1A and 2A, below. With reference to Scheme 1A, tryptophan is coupled with anhydride reagent 116, wherein X is Cl or F. The resulting trihalo oxazolone 118 is then exposed to AlCl₃ to facilitate cyclization to ketoamide 120. After treating ketoamide 120 with CuBr₂, diketone 122 is obtained, which can then undergo a Grignard addition reaction with magnesium bromide reagent 124 to provide coupled alpha-hydroxy ketone intermediate 126. Without being limited to a single operating mechanism, it current is believed that the regioselectivity of the addition to diketone 122 is expected to occur by first deprotonation of the enolizable position of the cyclopentadione, protecting the C—O ketone and permitting the selective addition to the desired C—O ketone. Subsequent dehydration to alkene monomer 128 is facilitate by reacting coupled alpha-hydroxy ketone intermediate 126 with SOCl₂ and pyridine. With reference to Scheme 2A, oxidative dimerization of alkene monomer 128 can be carried out by exposing alkene monomer 128 to Cu(OAc)₂ and heat. Other conditions for this step can be used, as described above (e.g., electrochemical-based dimerization and/or using other oxidants). In some aspects, products 206 and 208, which possess different oxidation states, can be produced via the dimerization step; however, each of these products can be converted to directly to scytonemin (210a of Scheme 2A, wherein R is H) upon exposure to DDQ or indirectly to scytonemin via intermediate 210b. In some other aspects, alkene monomer 128 can be dimerized and oxidized directly to intermediate 210b using Cu(OAc)₂ and heat.

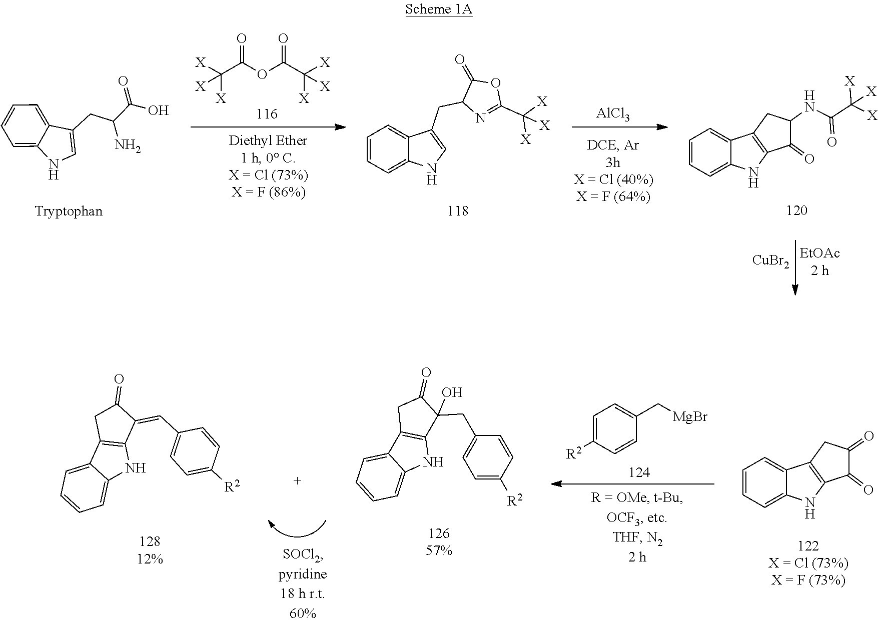

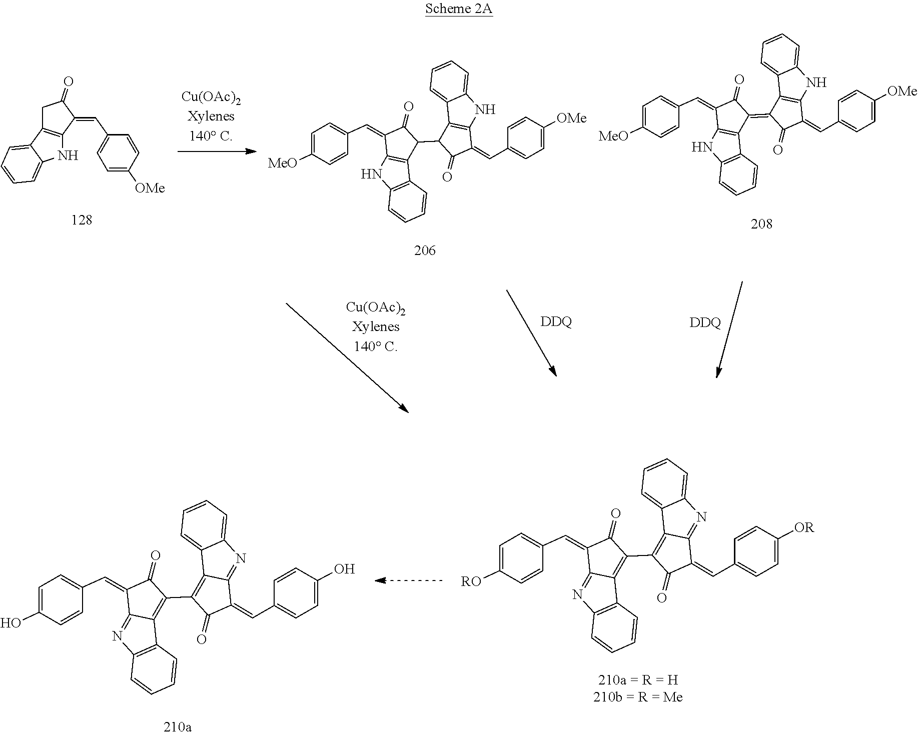

A representative method for making a scytonemin derivative according the method summarized in Scheme 3 is provided below in Scheme 3A. Scytonemin is exposed to aqueous ammonia in the presence of acetone at ambient temperature to produce scytonemin imine.

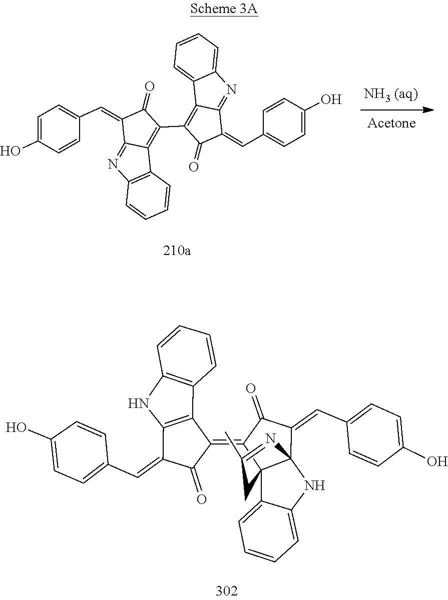

Using the method according to aspects of the present disclosure, scytonemin (including analogs and/or derivatives thereof) can be prepared more efficiently in comparison to conventional methods. Aspects of the method disclosed herein use inexpensive starting materials and fewer synthetic steps, which makes the method amenable to cost efficient scale-up. Additionally, structural analogs and/or derivatives of scytonemin can be obtained using the disclosed method, whereas such opportunities either do not exist or are inefficient using conventional methods.

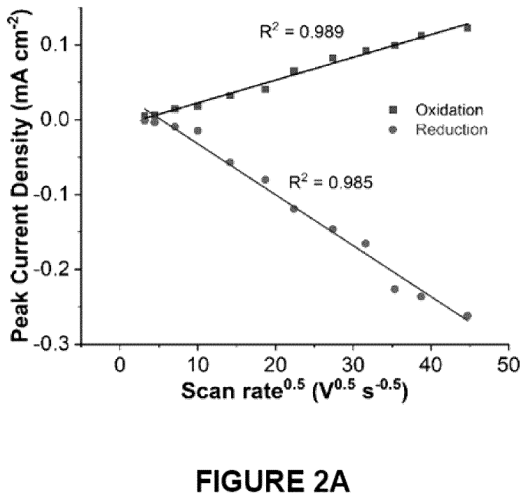

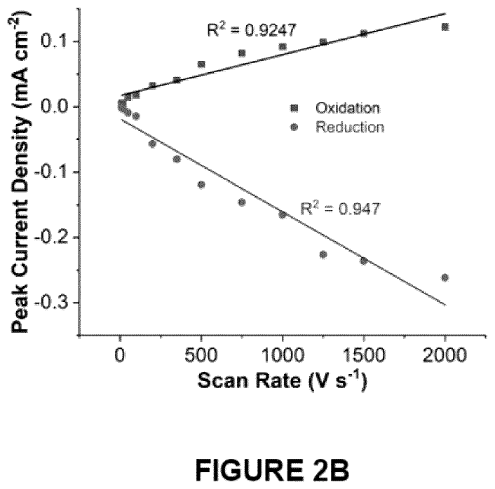

In particular aspects of the disclosure, the interconversion between the oxidized and reduced forms of scytonemin (including analogs and/or derivatives thereof) can be assessed. To do so, cyclic voltammetry analysis can be used. Other electrochemical parameters can be elucidated using CV. For example, using Randles-Sevcik analysis (e.g., see FIGS. 2A-2B), it was determined that scytonemin in solution follows what is expected for a diffusion-limited reversible electrochemical process; that is, the peak current densities for both oxidation and reduction are linear with the square root of the scan rate as opposed to being linear with the scan rate, the latter of which would occur for a surface-adsorbed species or irreversible process. Additionally, Laviron analysis allows for the calculation of electron transfer kinetics to and from scytonemin. In some aspects of the disclosure, the scytonemin can be immobilized to evaluate electrochemical properties in aqueous environments (as scytonemin is insoluble in water).

The redox properties of scytonemin described in the present disclosure suggest that it could be acting to quench oxygen/ROS extracellularly, shield UV across the spectrum thereby preventing direct UV damage and/or intracellular ROS production and damage. As such, it currently is believed that scytonemin (including analogs and/or derivatives thereof) made according to aspects of the method disclosed herein can be used in various applications. In some aspects, the compounds of the present disclosure can be used to protect against reactive oxygen species (“ROS”). In some particular aspects, the compounds can be used as a UV protectant (e.g., sunscreen or other protective barrier). In yet other aspects, the compounds can be used to treat or prevent ROS-induced damage. In yet some other aspects, the compounds can be used to reduce or prevent intracellular ROS production. Compounds according to the present disclosure can be used to treat and/or prevent diseases and/or disorders associated with UV damage and/or ROS-induced damage and/or intracellular ROS production. Therapeutically effective amounts of the compound (or a mixture of compounds) can be administered or applied to a subject to treat or prevent any such diseases/disorders. In some aspects, compounds according to the present disclosure can be used as anti-inflammatory and/or anti-proliferative therapeutics. For example, the compounds may inhibit skin inflammation by blocking inflammatory mediator expression, such as by down-regulation of NF-κB activity; or they may be used to inhibit proliferation of human fibroblasts and/or endothelial cells by selectively inhibiting kinases (e.g., human polo-like kinase 1, Myt1, cyclin-dependent kinase 1 (cyclin B), checkpoint kinase 1, and protein kinase C, and the like). In some aspects, the compounds may suppress human T-lymphoid Jurkat cell growth and/or LPS/IFNc-stimulated NO production in murine macrophage RAW264 cells. In yet additional aspects, the compounds may be used in material science applications. For example, compounds including R¹ groups that are of sufficient size or are positioned appropriately to inhibit rotation about the bond linking the monomer groups of any dimerized product may exhibit tunable electronic and/or magnetic properties and thus can be used in electronic materials applications.

Analogs and/or derivatives of scytonemin can include compounds according to Formulas IIA or IIB, shown below, including any pharmaceutically acceptable salts thereof. With reference to Formulas IIA and IIB, each R¹ independently is selected from aliphatic, heteroaliphatic, haloaliphatic, haloheteroaliphatic, aromatic, or an organic functional group; each R² independently is selected from aliphatic, heteroaliphatic, haloaliphatic, haloheteroaliphatic, aromatic, or an organic functional group; R³ is aliphatic, aromatic, or joins with the carbon atom alpha to the imine carbon atom of Formula IIB to form cyclic group (e.g., a 4-, 5-, 6-, or 7-membered ring); n is an integer selected from 0 to 4, and m is an integer selected from 0 to 5. In independent aspects, compounds of Formula IIA do not include, or are other than, scytonemin. In independent aspects, compounds of Formula IIB do not include, or are other than, scytonemin imine (wherein each n is 0, each m is 1, and each R² is OH). With reference to Formula IIB, any and all diastereomers are contemplated.

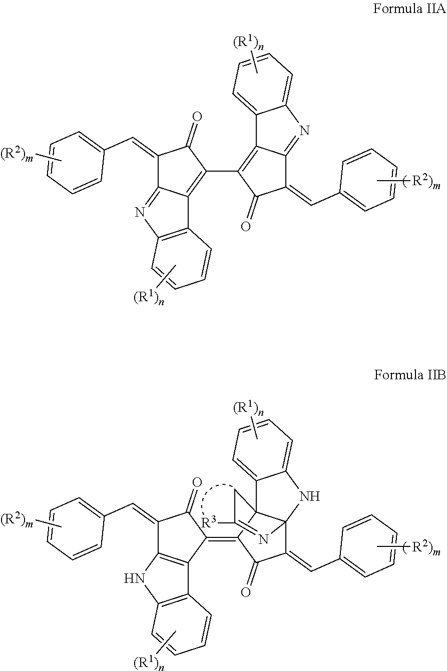

In some aspects of the disclosure, each R¹ independently can be selected from alkyl (e.g., lower alkyl, such as methyl, ethyl, propyl, butyl, pentyl, hexyl, heptyl, octyl, nonyl, or decyl), alkenyl, alkynyl, heteroalkyl (e.g., thioether, ether, methoxy, ethoxy, propoxy, butoxy, secondary amine, etc.), heteroalkenyl, heteroalkynyl, haloalkyl (e.g., CF₃ and the like), haloalkenyl, haloalkynyl, haloheteroalkyl (e.g., OCF₃ and the like), haloheteroalkenyl, haloheteroalkynyl, aryl, heteroaryl, or an organic functional group selected from OH, SH, NH₂, nitro, cyano, and the like. In some aspect, R¹ can be an ether group that is a polyalkylene oxide (e.g., a PEG group). In some aspects, each n is 1 and R¹ is positioned one, two, or three carbon atoms away from the phenyl carbon atom bearing the NH group of the illustrated indole.

In some aspects, each R² independently can be selected from alkyl (e.g., lower alkyl, such as methyl, ethyl, propyl, butyl, pentyl, hexyl, heptyl, octyl, nonyl, or decyl), alkenyl, alkynyl, heteroalkyl (e.g., methoxy, ethoxy, propoxy, butoxy, and the like), heteroalkenyl, heteroalkynyl, haloalkyl (e.g., CF₃ and the like), haloalkenyl, haloalkynyl, haloheteroalkyl (e.g., OCF₃ and the like), haloheteroalkenyl, haloheteroalkynyl, aryl, heteroaryl, or an organic functional group selected from OH, SH, NH₂, nitro, cyano, and the like. In some aspects, each m is 1 and R² is positioned meta, ortho, or para relative to the bond connecting the aryl ring to which R² is attached to the rest of the compound. In some aspect, R² can be an ether group that is a polyalkylene oxide (e.g., a PEG group). In exemplary aspects, each m is 1 and R² is positioned para relative to the bond connecting the aryl ring to which R² is attached to the rest of the compound. In such exemplary aspects, R² is selected from OMe, OCF₃, or t-butyl.

In some aspects, R³ is lower alkyl (e.g., methyl, ethyl, propyl, butyl, pentyl, hexyl, heptyl, octyl, nonyl, or decyl) or aryl (e.g., phenyl, naphthyl, or the like). In particular aspects, R³ is methyl, butyl, cyclohexyl, phenyl, 4-t-butylcyclohexyl, or R³ forms a cyclic ring with the carbon atom positioned alpha to the imine carbon atom as illustrated by the dashed arch in Formula IIB (e.g., a 4-, 5-. 6-, or 7-membered ring).

In particular aspects, the analog and/or derivative of scytonemin can be selected from any of the compounds illustrated below.

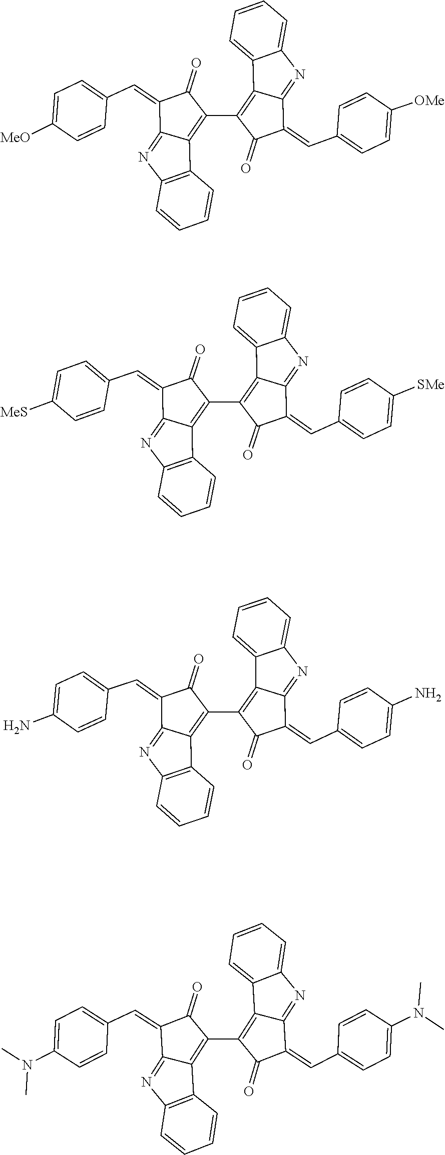

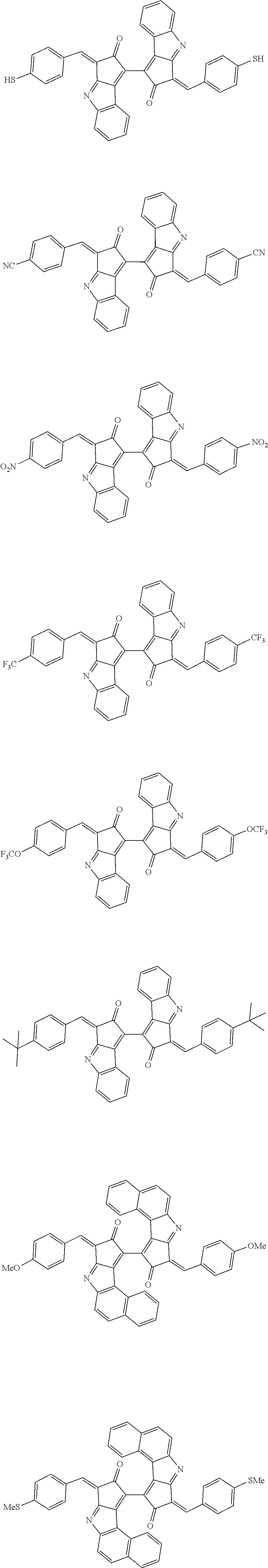

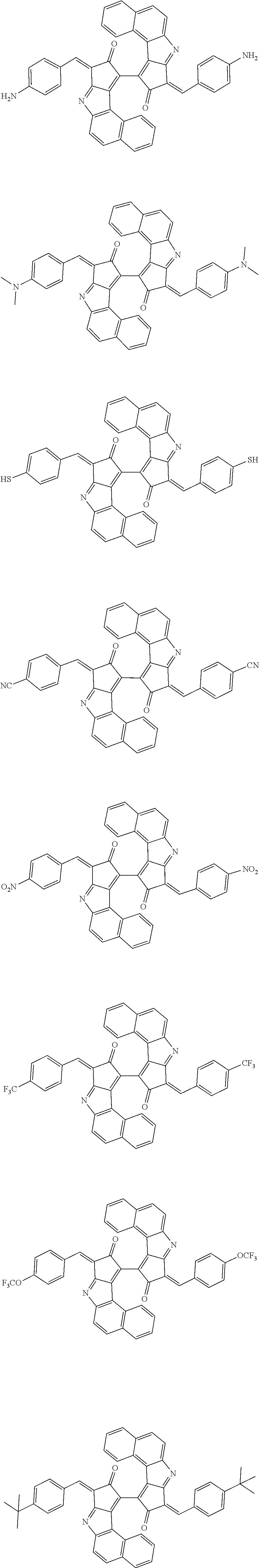

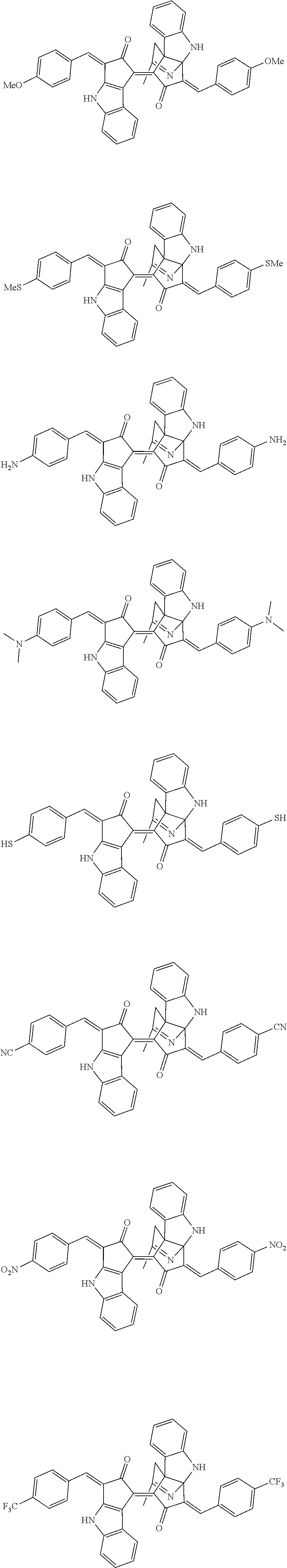

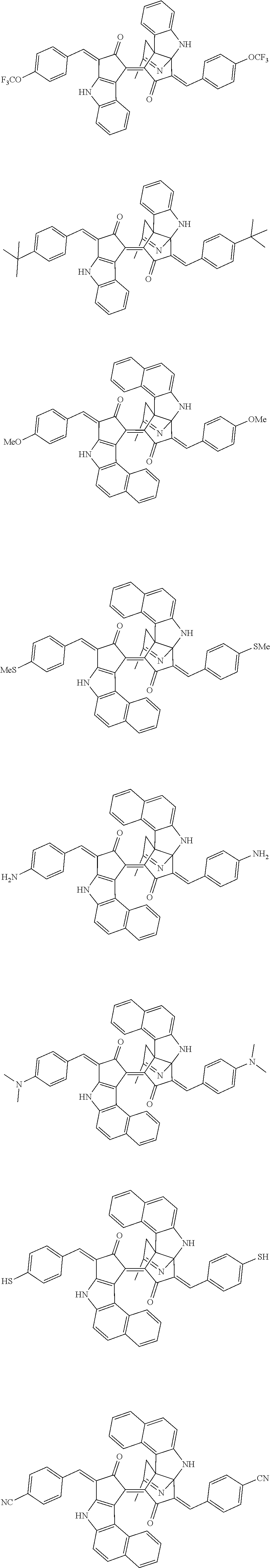

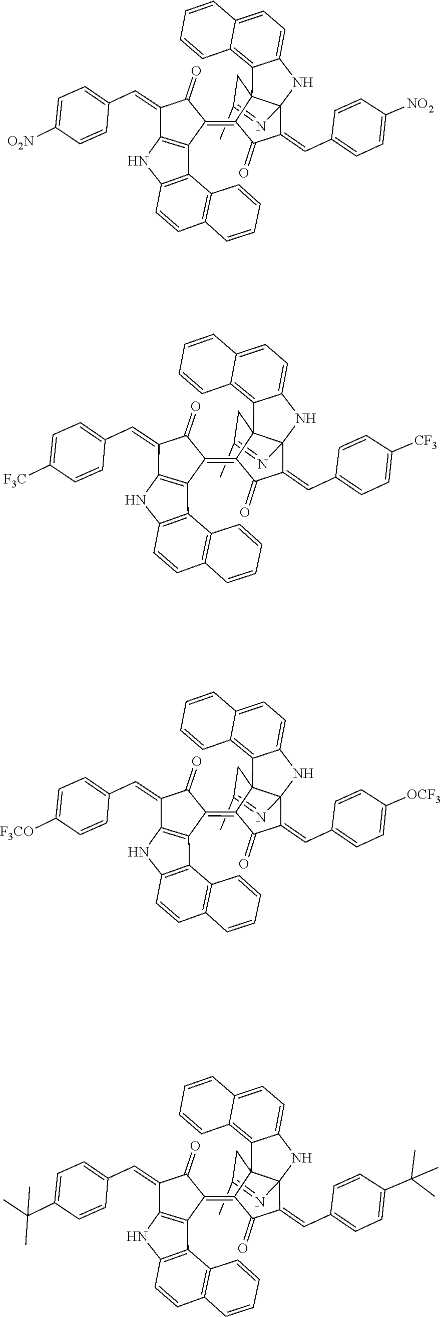 [SMILES: CC1=NC23Nc4ccc5ccccc5c4C2(C1)/C(=C1\C(=O)/C(=C/c2ccc([N+](=O)[O-])cc2)c2[nH]c4ccc5ccccc5c4c21)C(=O)/C3=C/c1ccc([N+](=O)[O-])cc1 ; CC1=NC23Nc4ccc5ccccc5c4C2(C1)/C(=C1\C(=O)/C(=C/c2ccc(C(F)(F)F)cc2)c2[nH]c4ccc5ccccc5c4c21)C(=O)/C3=C/c1ccc(C(F)(F)F)cc1 ; CC1=NC23Nc4ccc5ccccc5c4C2(C1)/C(=C1\C(=O)/C(=C/c2ccc(OC(F)(F)F)cc2)c2[nH]c4ccc5ccccc5c4c21)C(=O)/C3=C/c1ccc(OC(F)(F)F)cc1 ; CC1=NC23Nc4ccc5ccccc5c4C2(C1)/C(=C1\C(=O)/C(=C/c2ccc(C(C)(C)C)cc2)c2[nH]c4ccc5ccccc5c4c21)C(=O)/C3=C/c1ccc(C(C)(C)C)cc1]

Disclosed herein is a method for making scytonemin, or a scytonemin analog or a scytonemin derivative, the method including: converting a tryptophan-based starting material to an alkene monomer intermediate having a structure according to Formula I; dimerizing the alkene monomer intermediate by exposing the alkene monomer intermediate to dimerization conditions, wherein (i) dimerizing the alkene monomer intermediate produces the scytonemin analog; or (ii) dimerizing the alkene monomer intermediate produces a scytonemin compound precursor having a structure according to Formula II and the method can further include oxidizing the scytonemin compound precursor to provide scytonemin or the scytonemin analog; wherein Formulas I and II are as described herein and, wherein with respect to these formulas, each R¹ independently is selected from aliphatic, heteroaliphatic, haloaliphatic, haloheteroaliphatic, aromatic, or an organic functional group, or two R¹ groups join together to form a fused ring system with carbon atoms to which the two R¹ groups are bound; each R² independently is selected from aliphatic, heteroaliphatic, haloaliphatic, haloheteroaliphatic, aromatic, or an organic functional group, or two R² groups join together to form a fused ring system with carbon atoms to which the two R² groups are bound; n is an integer selected from 0 to 4; and m is an integer selected from 0 to 5.

In any or all of the above aspects, converting the tryptophan-based starting material to the alkene monomer intermediate includes: (i) coupling the tryptophan-based starting material with an anhydride reagent according to Formula III as described herein to provide a trihalo oxazolone (wherein, with respect to Formula III, each X independently is a halogen); (ii) exposing the trihalo oxazolone to a Lewis acid to provide a ketoamide intermediate according to Formula IV as described herein (wherein, with respect to Formula IV, each X independently is a halogen; R¹ is selected from aliphatic, heteroaliphatic, haloaliphatic, haloheteroaliphatic, aromatic, or an organic functional group; and n is an integer selected from 0 to 4); (iii) oxidizing the ketoamide intermediate to a diketone intermediate; (iv) performing a Grignard addition reaction to functionalize the diketone intermediate at a position bearing a ketone of the diketone intermediate to provide an alpha-hydroxy ketone-containing compound; and (v) dehydrating the alpha-hydroxy ketone-containing compound to provide the alkene monomer intermediate.

In any or all of the above aspects, the tryptophan-based starting material has a structure according to Formula V as described herein.

In any or all of the above aspects, the anhydride reagent is selected from

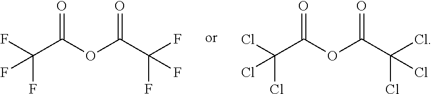 [SMILES: O=C(OC(=O)C(F)(F)F)C(F)(F)F ; "or" ; O=C(OC(=O)C(Cl)(Cl)Cl)C(Cl)(Cl)Cl]

In any or all of the above aspects, the trihalo oxazolone is selected from

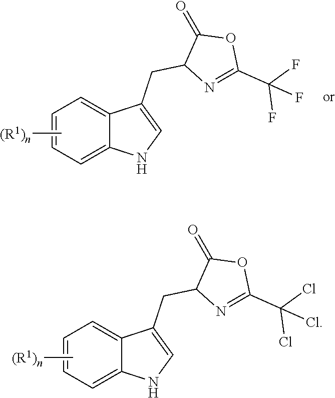

In any or all of the above aspects, the Lewis acid is selected from AlCl₃, BF₃·OEt₂, FeCl₃, methanesulfonic acid, or polyphosphoric acid.

In any or all of the above aspects, the ketoamide intermediate is selected from

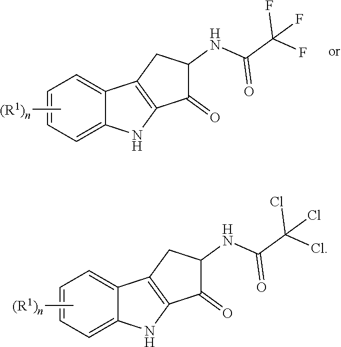

In any or all of the above aspects, oxidizing the ketoamide intermediate to the diketone intermediate includes using CuBr₂, Br₂, N-bromosuccinimide, I₂, PhI(OAc)₂, or tBuOCl.

In any or all of the above aspects, the diketone intermediate has a structure according to Formula VI as described herein.

In any or all of the above aspects, performing the Grignard addition reaction includes exposing the diketone intermediate to a reagent having a structure according to a Formula VII as described herein (wherein, with respect to Formula VII, each R² independently is selected from aliphatic, heteroaliphatic, haloaliphatic, haloheteroaliphatic, aromatic, or an organic functional group, and m is an integer selected from 0 to 5).

In any or all of the above aspects, the alpha-hydroxy ketone-containing compound has a structure according to Formula VIII as described herein (wherein, with reference to Formula VIII, each R¹ independently is selected from aliphatic, heteroaliphatic, haloaliphatic, haloheteroaliphatic, aromatic, or an organic functional group, or two R¹ groups join together to form a fused ring system with carbon atoms to which the two R¹ groups are bound; R² independently is selected from aliphatic, heteroaliphatic, haloaliphatic, haloheteroaliphatic, aromatic, or an organic functional group, or two R² groups join together to form a fused ring system with carbon atoms to which the two R² groups are bound; n is an integer selected from 0 to 4; and m is an integer selected from 0 to 5).

In any or all of the above aspects, dehydrating the alpha-hydroxy ketone-containing compound includes exposing the alpha-hydroxy ketone-containing compound to (i) SOCl₂ and pyridine, or (ii) a Bronsted acid selected from para-toluene sulfonic acid, or sulfuric acid.

In any or all of the above aspects, the dimerization conditions include using an oxidant selected from Cu(OAc)₂, AgF, PhI(OAc)₂, I₂, K₃[Fe(CN)₆], or FeCl₃.

In any or all of the above aspects, the electrochemical reaction conditions include exposing the alkene monomer intermediate to a basic methanol solution in an electrochemical cell including a working electrode and a counter electrode operated at an operating potential.

In any or all of the above aspects, oxidizing the scytonemin compound precursor includes using 2,3-dichloro-5,6-dicyanobenzoquinone.

In any or all of the above aspects, the scytonemin analog has a structure according to Formula IIA as described herein, provided that at least one R² is not OH.

In any or all of the above aspects, each R¹ independently is selected from alkyl, alkenyl, alkynyl, heteroalkyl, heteroalkenyl, heteroalkynyl, haloalkyl, haloalkenyl, haloalkynyl, haloheteroalkyl, haloheteroalkenyl, haloheteroalkynyl, aryl, heteroaryl, or an organic functional group selected from OH, SH, NH₂, nitro, or cyano, or wherein two R¹ groups join together to form a 6-membered aromatic ring fused with the phenyl ring to which the two R¹ group are bound.

In any or all of the above aspects, each R² independently is selected from alkyl, alkenyl, alkynyl, heteroalkyl, heteroalkenyl, heteroalkynyl, haloalkyl, haloalkenyl, haloalkynyl, haloheteroalkyl, haloheteroalkenyl, haloheteroalkynyl, aryl, heteroaryl, or an organic functional group selected from OH, SH, NH₂, nitro, or cyano.

In any or all of the above aspects, n is 0 and each m is 1.

In any or all of the above aspects, each R² is selected from OH, OMe, OCF₃, or t-butyl.

In any or all of the above aspects, the scytonemin analog is selected from

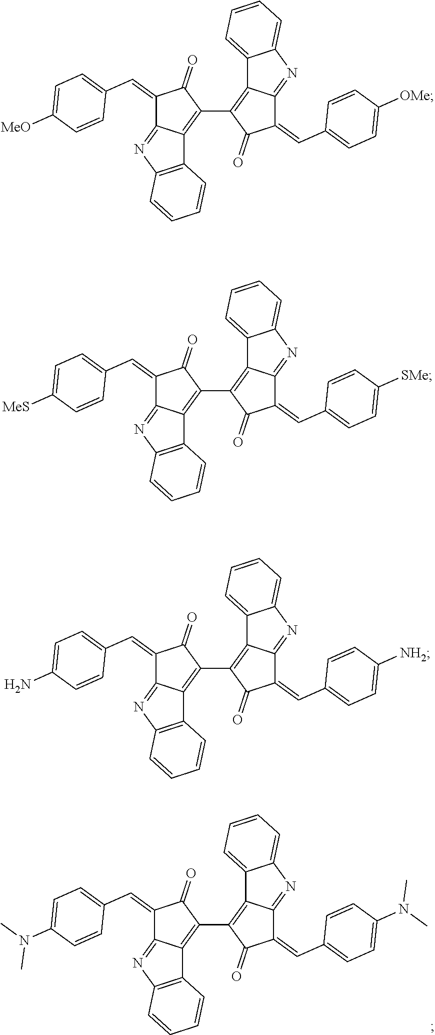

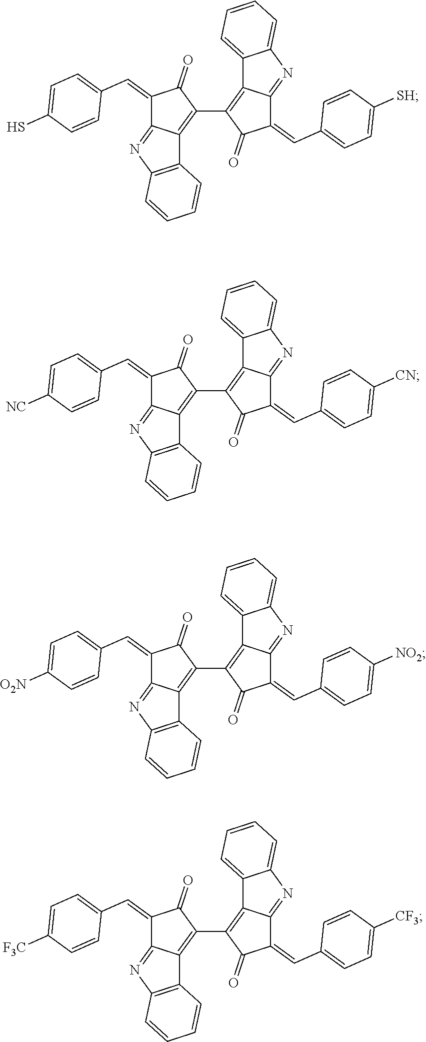 [SMILES: O=C1C(C2=C3C(=Nc4ccccc43)/C(=C\c3ccc(S)cc3)C2=O)=C2C(=Nc3ccccc32)/C1=C\c1ccc(S)cc1 ; N#Cc1ccc(/C=C2/C(=O)C(C3=C4C(=Nc5ccccc54)/C(=C\c4ccc(C#N)cc4)C3=O)=C3C2=Nc2ccccc23)cc1 ; O=C1C(C2=C3C(=Nc4ccccc43)/C(=C\c3ccc([N+](=O)[O-])cc3)C2=O)=C2C(=Nc3ccccc32)/C1=C\c1ccc([N+](=O)[O-])cc1 ; O=C1C(C2=C3C(=Nc4ccccc43)/C(=C\c3ccc(C(F)(F)F)cc3)C2=O)=C2C(=Nc3ccccc32)/C1=C\c1ccc(C(F)(F)F)cc1]

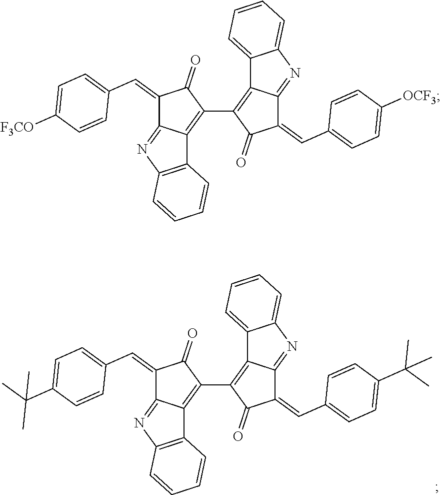 [SMILES: O=C1C(C2=C3C(=Nc4ccccc43)/C(=C\c3ccc(OC(F)(F)F)cc3)C2=O)=C2C(=Nc3ccccc32)/C1=C\c1ccc(OC(F)(F)F)cc1 ; CC(C)(C)c1ccc(/C=C2/C(=O)C(C3=C4C(=Nc5ccccc54)/C(=C\c4ccc(C(C)(C)C)cc4)C3=O)=C3C2=Nc2ccccc23)cc1]

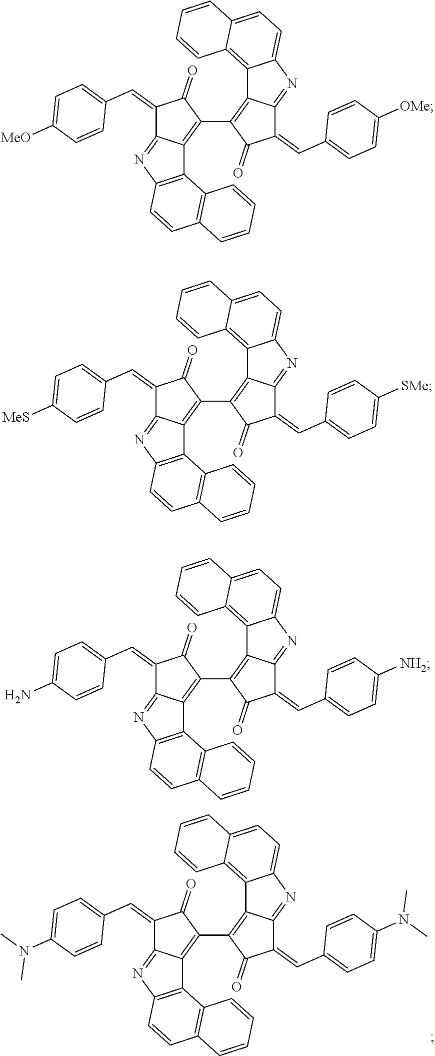

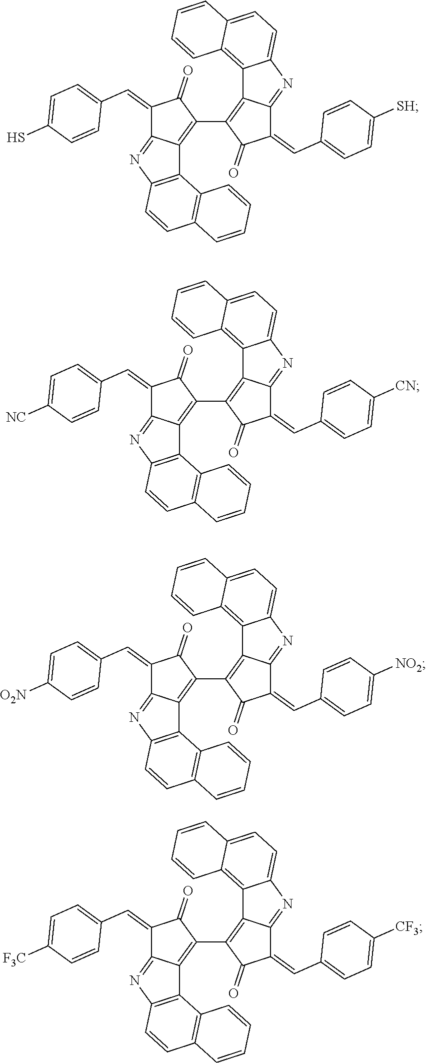

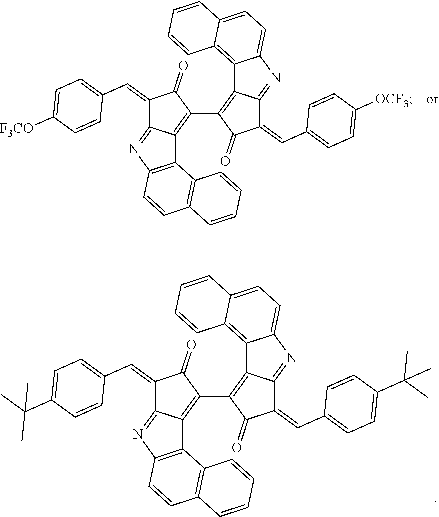 [SMILES: O=C1C(C2=C3C(=Nc4ccc5ccccc5c43)/C(=C\c3ccc(OC(F)(F)F)cc3)C2=O)=C2C(=Nc3ccc4ccccc4c32)/C1=C\c1ccc(OC(F)(F)F)cc1 ; CC(C)(C)c1ccc(/C=C2/C(=O)C(C3=C4C(=Nc5ccc6ccccc6c54)/C(=C\c4ccc(C(C)(C)C)cc4)C3=O)=C3C2=Nc2ccc4ccccc4c23)cc1]

In any or all of the above aspects, the method can further include exposing the scytonemin or the scytonemin analog to aqueous ammonia in the presence of a carbonyl-containing reactant to form a scytonemin derivative having a structure according to Formula IIB as described herein, wherein R³ is aliphatic, aromatic, or joins with the carbon atom alpha to the imine carbon atom of Formula IIB to form a cyclic group.

In any or all of the above aspects, the scytonemin derivative is scytonemin imine.

Also disclosed is a compound having a formula according to Formula IIA or Formula IIB as described herein, wherein each R¹ independently is selected from aliphatic, heteroaliphatic, haloaliphatic, haloheteroaliphatic, aromatic, or an organic functional group; each R² independently is selected from aliphatic, heteroaliphatic, haloaliphatic, haloheteroaliphatic, aromatic, or an organic functional group; R³ is aliphatic, aromatic, or joins with the carbon atom alpha to the imine carbon atom of Formula IIB to form cyclic group; n is an integer selected from 0 to 4, and m is an integer selected from 0 to 5; provided that the compound is not scytonemin or scytonemin imine.

### EXAMPLES

In order to provide a better understanding of the foregoing discussion, the following non-limiting examples are offered. Although the examples can be directed to specific embodiments, they are not to be viewed as limiting the invention in any specific respect.

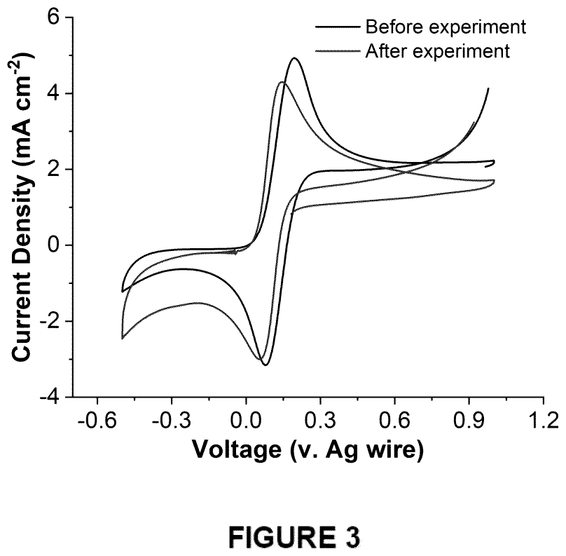

Chemicals used for electrochemical experiments (NaClO₄, H₃BO₃, H₃PO₄, CH₃OOOH, ferrocene, acetonitrile, DMSO) were obtained from commercial sources and used without purification. All electrochemical studies were performed using a VSP-300 Biologic potentiostat unless stated otherwise. For experiments conducted with analytes in solution, cyclic voltammetry of scytonemin, curcumin, and daidzen were performed with 5 mM of the analyte and 50 mM NaClO₄ in acetonitrile (MeCN). A three-electrode cell was utilized, in which glassy carbon served as the working electrode, Ag wire served as the quasi-reference electrode, and a Pt wire serves as the counter electrode. The Ag wire reference electrode was calibrated with respect to a ferrocene redox couple before and after each analyte experiment (FIG. 3 shows representative ferrocene CVs).

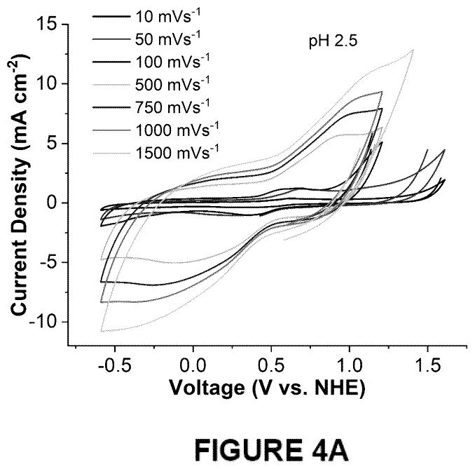

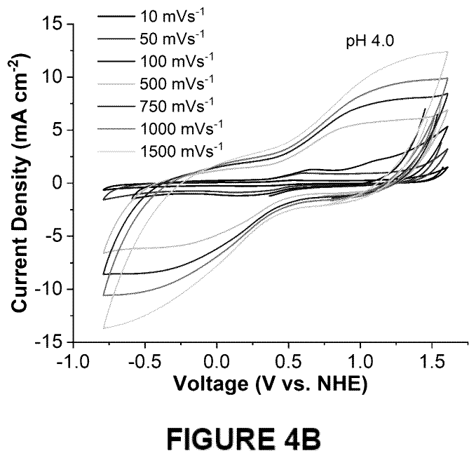

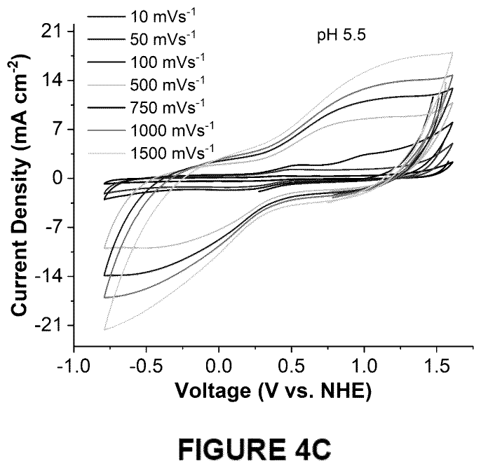

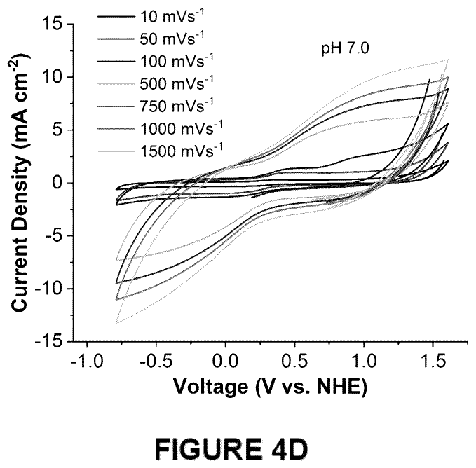

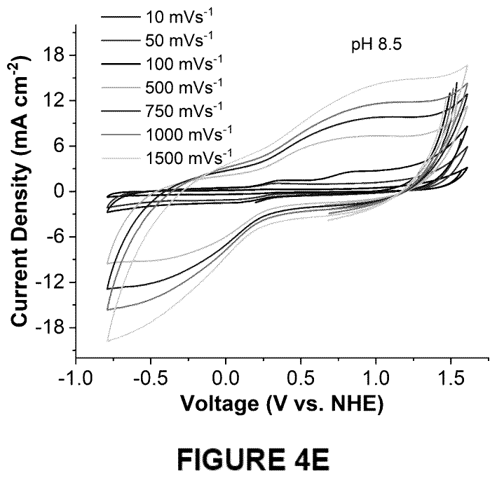

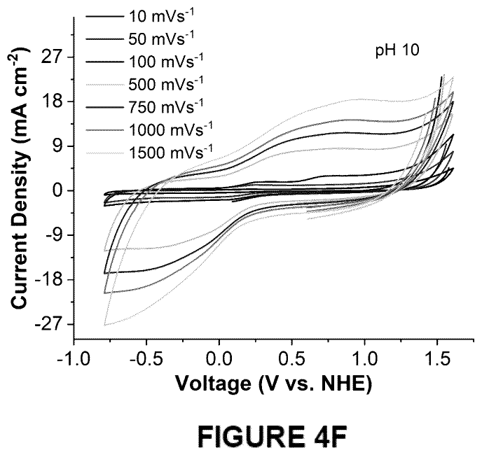

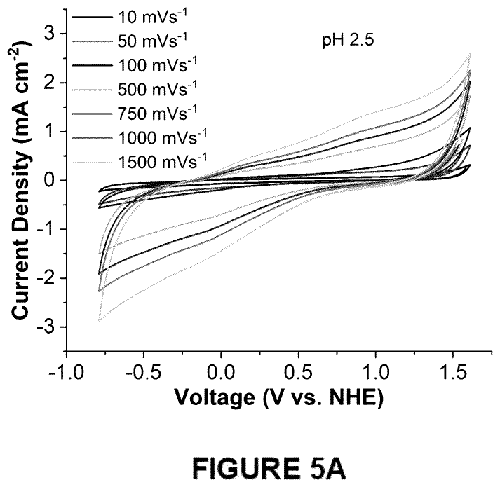

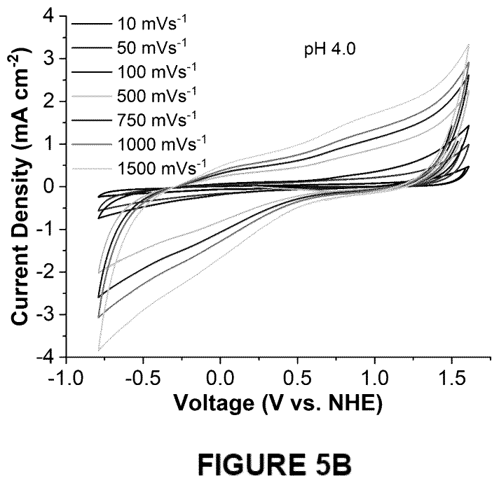

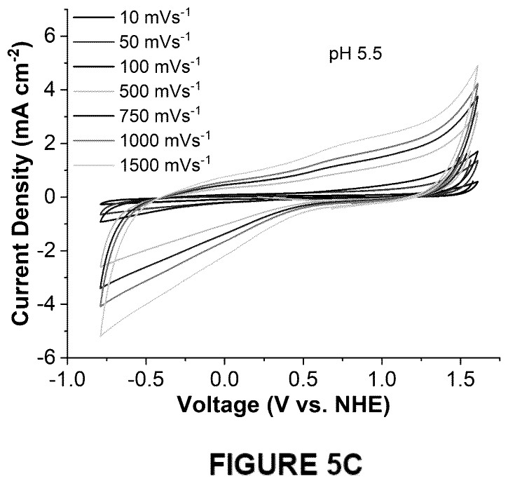

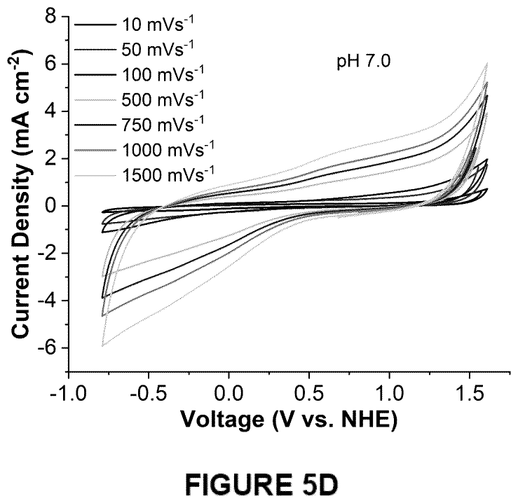

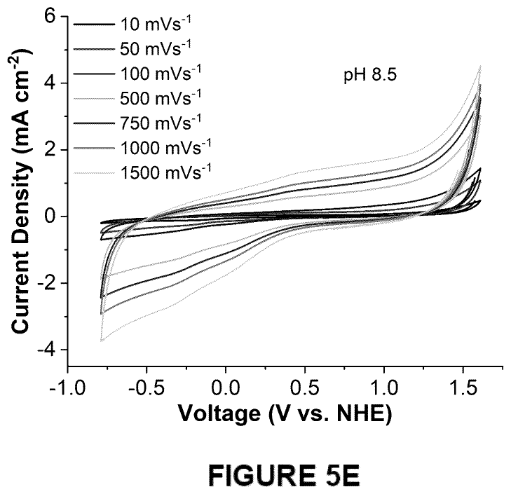

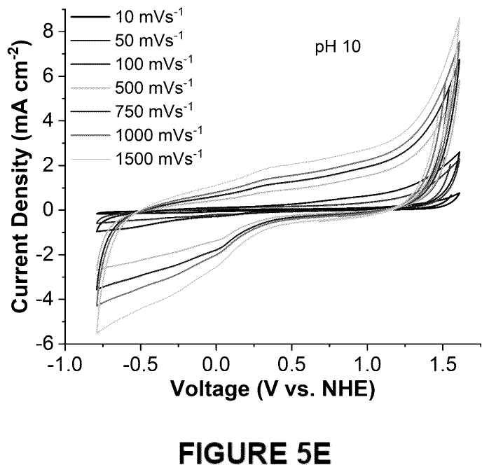

For experiments in aqueous electrolytes, Britton-Robinson buffers (40 mM H₃BO₃, 40 mM H₃PO₄, 40 mM CH₃COOH) were used and adjusted to the desired pH using NaOH. Glassy carbon (3 mm diameter) served as the working electrode and was polished using a suspension of 0.05 μm alumina followed by sonication for 8 min in water prior to use. A leakless Ag/AgCl/3 M KCl electrode (eDaq, Inc.) was used as the reference electrode, and a graphite road was used as the counter electrode. A freshly prepared ink was used to modify the glassy carbon electrode with scytonemin. The ink consisted of 1.2 mg scytonemin, 1.2 mg Vulcan XC-72, 2.2 mL MeOH, and 20 □L Nafion®. This mixture was sonicated for 15 minutes. The ink (60 □L) was then dropcast on the glassy carbon electrode in open air using a custom-built rotator (8 rpm) to promote uniform film formation. Results for CV of scytonemin in 1.2 M Britton-Robinson buffer are shown in FIGS. 4A-4F. Results for CV of di-O-methyl scytonemin in 1.2 M Britton-Robinson buffer are shown in FIGS. 5A-5E.

Spectroelectrochemistry was conducted using a solution containing 5 mM of scytonemin and 50 mM NaClO₄ in DMSO using a quartz cuvette with a 1 cm path length. Tin-doped indium oxide on glass (Xin Yan, Inc., 10 Ω/sq) served as the transparent working electrode in this experiment to measure the transmittance of the solution. An Ocean Optics FLAME-S—VIS-NIR spectrometer with an Ocean Insight HL2000-FHSA light source was used for transmission measurements. The spectrometer used a two-point calibration with the first point with the light source off (0% transmission) and the second point with the light source on and a cuvette containing the electrolyte solution without scytonemin (100% transmission).

To test the oxygen reduction activity of scytonemin using rotating ring-disk electrochemistry, a four-electrode cell was utilized, in which modified glassy carbon (5 mm diameter) served as the disk working electrode, a Pt ring served as the ring electrode, a graphite rod functioned as the counter electrode, and an Ag/AgCl/3 M KCl (eDaq, Inc.) electrode was used as the counter electrode. The glassy carbon working electrode was modified with scytonemin using the ink formulation described above except 100 QL of ink were dropcast to accommodate the larger electrode. The Pt ring electrodes (Pine Research Instrumentation, Inc.) were cleaned electrochemically in 15 mL of 0.1 M HClO₄ solution by cycling from −0.4 to +1.7 V vs Ag/AgCl at 100 mV/s until the oxide stripping at about 0.35 V remained constant. The electrolyte consisted of 45 mL of Britton-Robinson buffer solution and was sparged with O₂ for at least 10 minutes. During RRDE, the ring voltage was held at +1.5 V vs. Ag/AgCl. The number of electrons transferred to O₂ on the disc was calculated from the ring current using the collection efficiency obtained from an unmodified glassy carbon electrode as is standard practice.

### Example 1

In this example, tryptophan is converted to monomer 128 using the steps and conditions illustrated in Scheme 1A below.

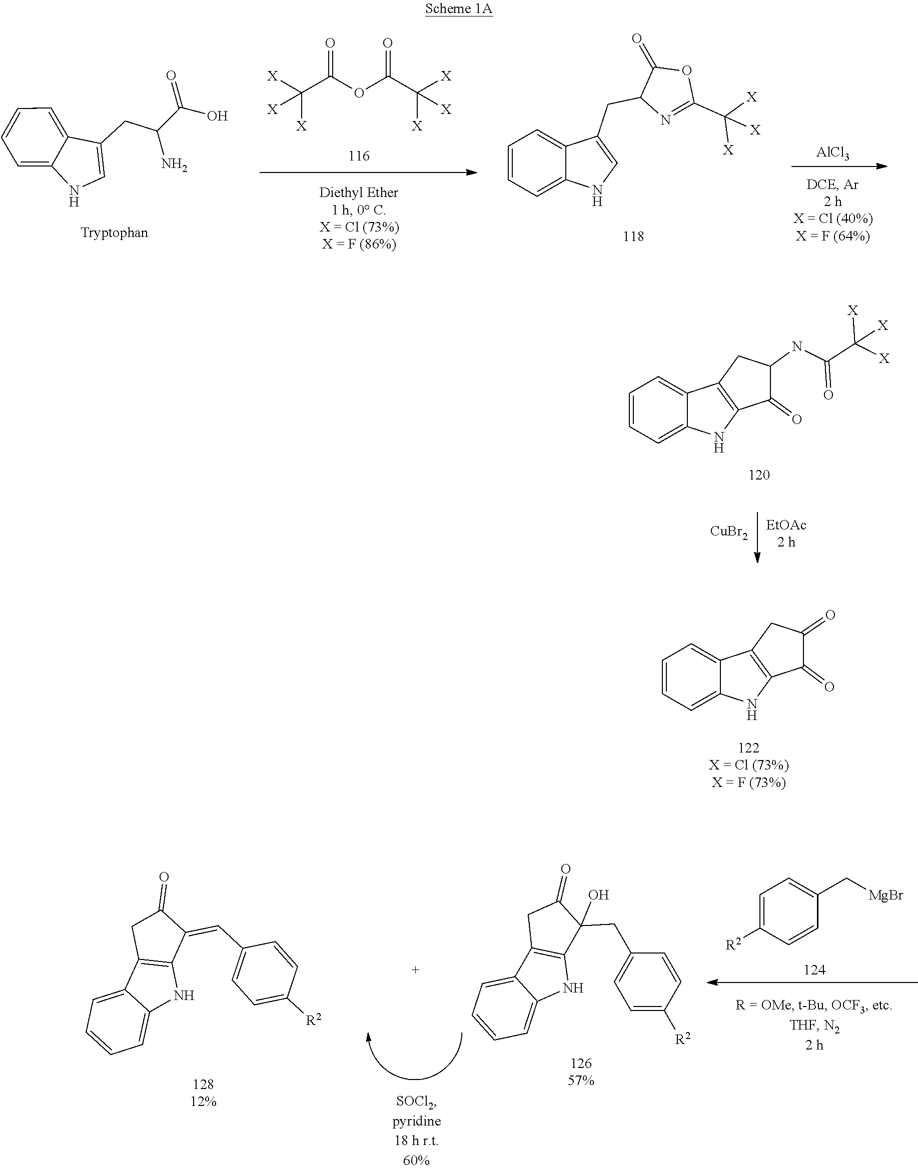

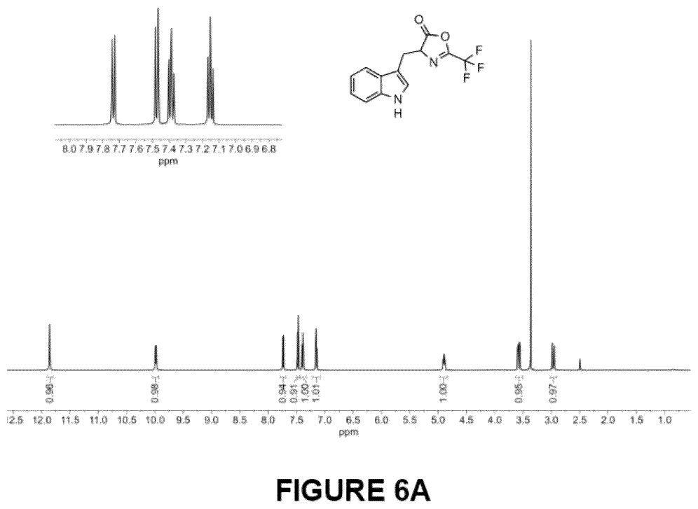

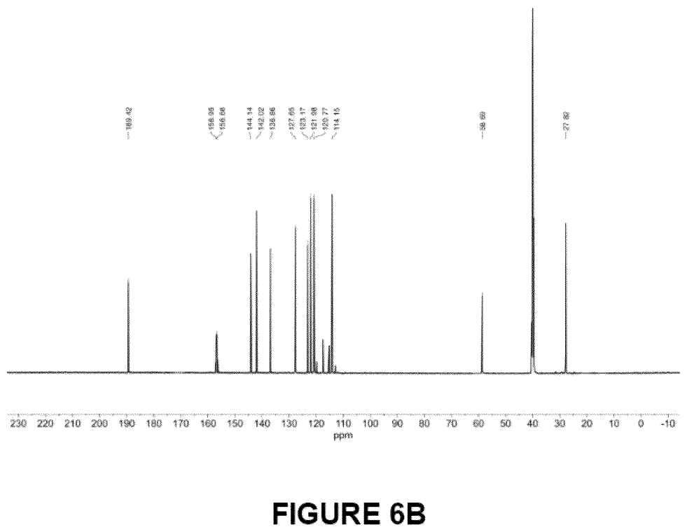

Trifluoro Oxazolone 118 (X=F)—Tryptophan (10.2 g, 50 mmol) was added to trifluoroacetic anhydride (16.6 mL, 120 mmol) in ether (100 mL) at 0° C. and the reaction started to heat up. Once the reaction cooled down it was removed from ice and left to stir at room temperature. Once the solid has gone fully into solution stirring was ceased and the reaction flask was put in the freezer overnight to form a precipitate. The precipitate was filtered, and hexanes was added to the mother liquor and the ether was evaporated off until a cloudy solution formed and was filtered again. 12.2 g (86%) of solid was recovered and used without further purification. (+/−)-HRESIMS 282.27905 M/z. ¹H and ¹³C-NMR spectra are shown in FIGS. 6A-6B.

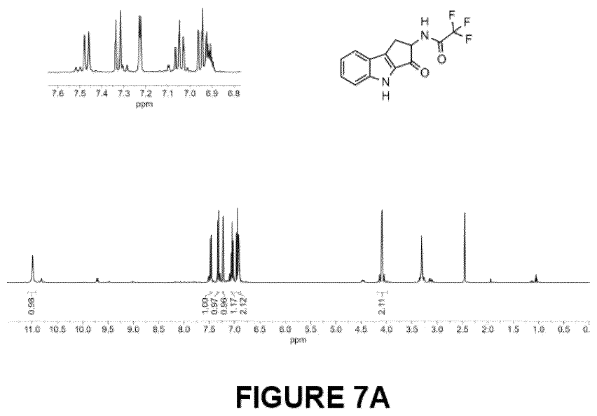

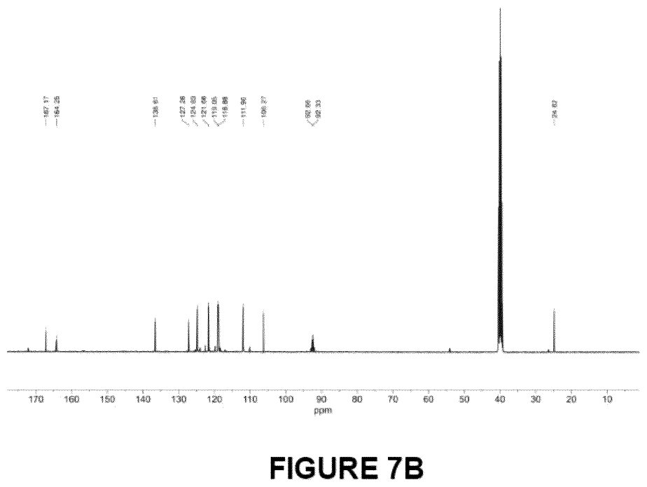

Ketoamide 120—Oxazolone 118 (6.2 g, 22 mmol) was added all at once to a stirred mixture of aluminum chloride (5.8 g, 44 mmol) in degassed dichloroethane (161 mL), and stirring was continued at room temperature under nitrogen for 2 hours. Reaction was quenched with sodium potassium tartrate and EtOAc was added until solution separated into two layers after mixing. Then liquid separation was performed, the organic layer dried with MgSO₄ and concentrated under vacuum. The resulting solid was dissolved in a minimal amount of EtOAc and then hexanes was added until a cloudy white precipitate formed, and the solution was put in the freezer. The solid was filtered, yielding 4.03 grams (65%) and used without further purification. ¹H NMR (500 MHz, DMSO-d₆) δ 11.86 (s, 1H), 9.98 (d, J=8.3 Hz, 1H), 7.74 (dd, J=8.0, 1.0 Hz, 1H), 7.52-7.44 (m, 1H), 7.39 (ddd, J=8.3, 7.0, 1.2 Hz, 1H), 7.15 (ddd, J=8.0, 6.9, 1.0 Hz, 1H), 4.90 (ddd, J=8.3, 6.9, 3.3 Hz, 1H), 3.57 (dd, J=16.3, 7.0 Hz, 1H), 2.97 (dd, J=16.2, 3.4 Hz, 1H). ¹³C NMR (126 MHz, DMSO) δ 189.42, 156.95, 156.66, 144.14, 142.02, 136.86, 127.65, 123.17, 121.98, 120.77, 114.15, 58.69, 27.82. See FIGS. 7A and 7B. (+/−)-HRESIMS 283.06947 M/z.

Diketone 122—Copper(II) bromide (6.18 g, 27.7 mmol) was added, all at once, to a refluxing solution of ketoamide 120 (3.25 g, 11.5 mmol) in ethyl acetate (237 mL). Heating at reflux was continued for 2 hours. The mixture was cooled, and then washed with a dilute NaCl solution, then brine, dried (MgSO₄), and the solvents were evaporated in vacuo to give a solid.

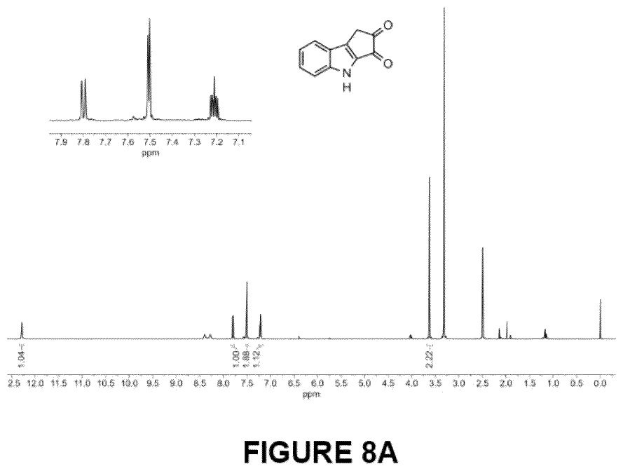

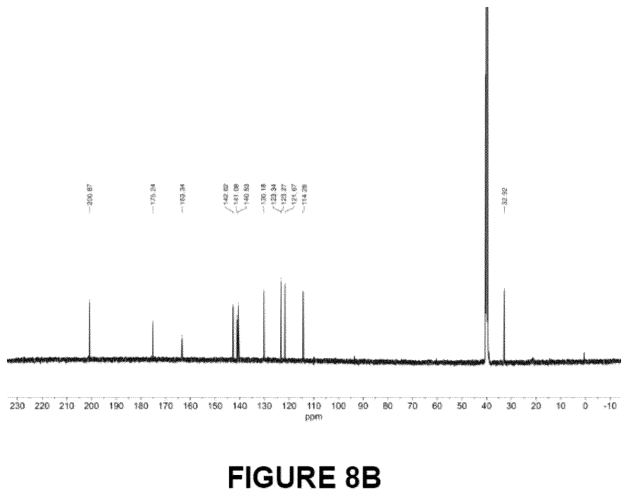

Solid was dissolved in small amount of methanol and then ether was added and put into the freezer. Solid was filtered giving pure diketone. Crystallization was repeated until no solid formed. Yielded 3.57 g (70%)¹H NMR (500 MHz, DMSO-d₆) δ 12.28 (s, 1H), 7.80 (dd, J=8.1, 0.9 Hz, 1H), 7.53-7.46 (m, 2H), 7.21 (ddd, J=8.0, 4.5, 3.4 Hz, 1H), 3.63 (s, 2H). ¹³C NMR (126 MHz, DMSO) δ 200.87, 175.24, 163.34, 142.62, 141.08, 140.53, 130.18, 123.34, 123.27, 121.67, 114.28, 32.92. See FIGS. 8A-8B. (+/−)-HRESIMS 186.05525 M/z.

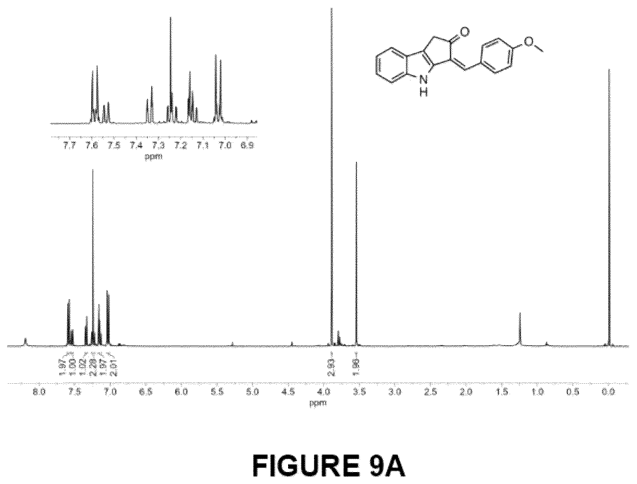

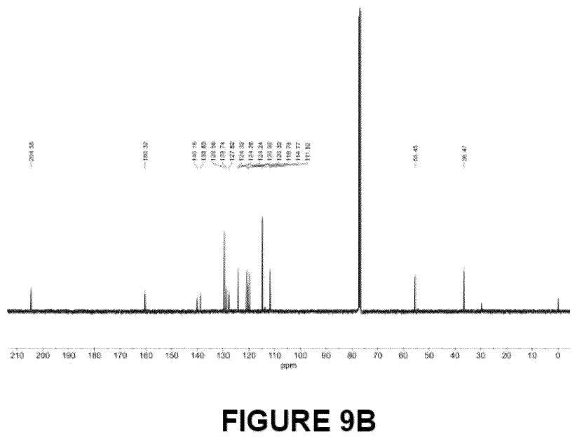

Methoxy Alkene 128—Diketone 122 (1.11 mg, 6 mmol) solution in THF (111 mL) was added to a solution of 4-methoxybenzyl magnesium chloride (48 mL, 12 mmol, 0.25M) at −78 C and let warm to room temperature and left to react for 4 hours. After which the reaction was quenched at room temp with 3M HCl and extracted with EtOAc (3×50 mL), then the organic layers were combined and washed with Brine and dried with NaSO₄. The alkene 128 was purified using flash chromatography eluting around 3:1 Hex:EtOAc, yielding 525 mg (30%)¹H NMR (400 MHz, Chloroform-d) δ 7.61-7.57 (m, 1H), 7.56-7.51 (m, OH), 7.37-7.31 (m, 1H), 7.27-7.21 (m, 1H), 7.16 (s, 1H), 7.15-7.12 (m, OH), 7.03 (d, J=8.7 Hz, 1H), 3.89 (s, 2H), 3.54 (s, 1H). ¹³C NMR (101 MHz, CDCl₃) δ 204.55, 160.32, 140.16, 138.83, 129.56, 128.74, 127.82, 124.32, 124.26, 124.24, 120.92, 120.32, 119.78, 114.77, 111.82, 55.45, 36.47. See FIGS. 9A-9B. (+/−)-HRESIMS 290.11811 M/z.

### Example 2

In this example, methoxy alkene 128 is converted to dimer 210 using the steps and conditions illustrated in Scheme 2A below.

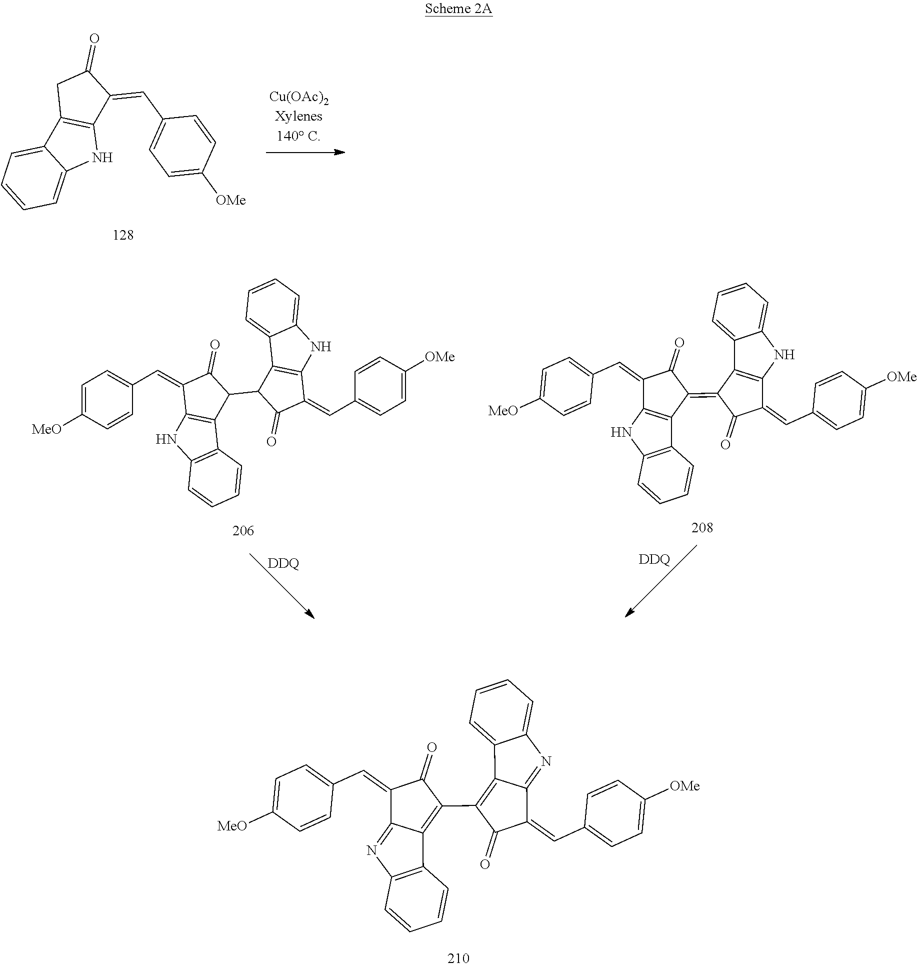

Methoxy Dimer 206 and/or 208—Methoxy alkene 128 (60 mg, 0.21 mmol) and Cu(OAc)₂ (41 mg, 0.21 mmol) in xylene (0.63 mL) was stirred under reflux at 140° C. for 4 hours. After 4 hours, the crude mixture including dimers 206 and/or 208 was filled through a pad of silica and concentrated down. The product was carried on to the next step without further purification.

Methoxy Scytonemin 210—The product from the first step of Scheme 1A (57 mg, 0.1 mmol) was dissolved in THF (2 mL) and DDQ (68.1 mg, 0.3 mmol) in THF (1 mL) was added, and the solution was stirred for 45 minutes. Then it was concentrated down and purified with preparative HPLC (H₂O:MeCN) to yield methoxy scytonemin 210 at 9 mg. Confirmed via HR-MS 572.17 M/z.

### Example 3

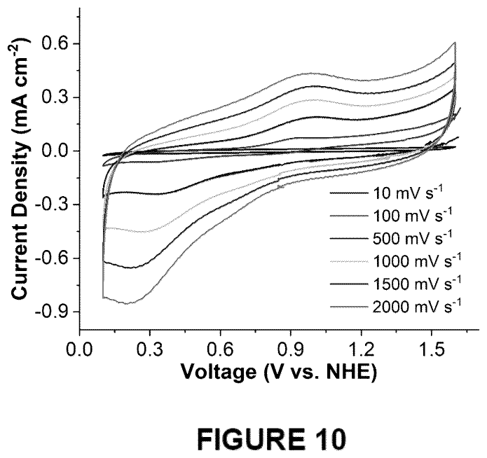

In this example, cyclic voltammetry was performed with an as-synthesized reduced form of scytonemin in acetonitrile with a NaClO₄ supporting electrolyte. Results for this particular example are shown in FIG. 10. At the various scan rates evaluated, the CVs exhibit one cathodic and one anodic peak, corresponding to the reduced and oxidized forms of scytonemin, respectively. In these CVs, the negative-going scan (reduction) is performed first, and despite the fact that the as-synthesized form is nominally reduced, a reduction peak is still observed on the first scan. It was determined that the as-synthesized material includes a mixture of oxidized and reduced forms as the reduced form is prone to aerial oxidization. Protons required for the redox process may come from adventitious water in the wet acetonitrile electrolyte (c.a. ˜0.1%). Additionally, Laviron analysis was used to calculate electron transfer kinetics to and from scytonemin. The cathodic and anodic electron transfer rates in the acetonitrile electrolyte were 1.4 s⁻¹ and 32 s⁻¹, respectively.

### Example 4

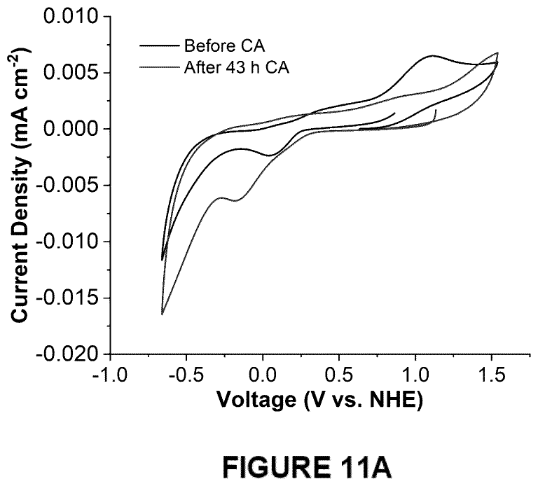

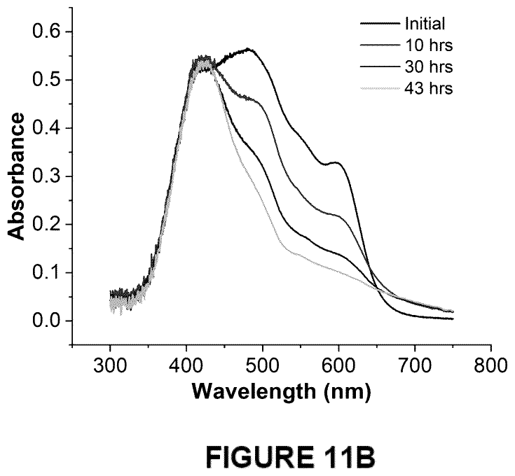

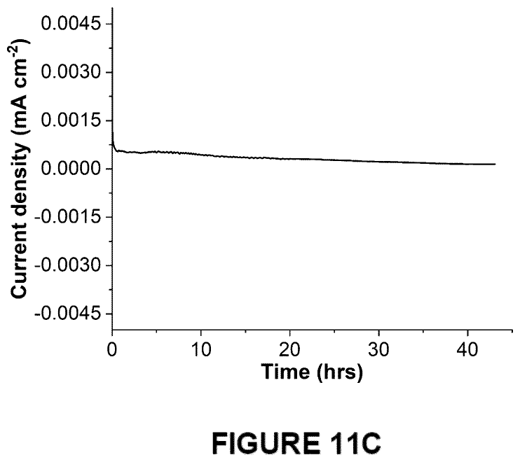

In this example, CV of scytonemin in DMSO on a transparent indium tin oxide on glass electrode also possessed cathodic and anodic peaks (FIG. 11A; line labeled “before CA”). The transparency of the electrode allows for spectroelectrochemical measurements during anodic chronoamperometry to convert the reduced scytonemin to its oxidized form. The UV-visible spectrum of the initial reduced scytonemin solution contains three absorption peaks at 420 nm, 480 nm, and 600 nm. In some aspects, the two lower energy peaks disappear, and the solution changes from brown to yellow over the course of 43 hours of chronoamperometry at +1.245 V vs. NHE (UV spectra provided by FIG. 11B and the chronoampermetric trace during oxidation provided by FIG. 11C). In particular aspects, a CV after 43 hours of chronoamperometry does not exhibit a significant anodic peak, indicating that this procedure mostly converts the reduced scytonemin to its oxidized form (FIG. 11A, line labeled “after 43 h CA”).

### Example 5

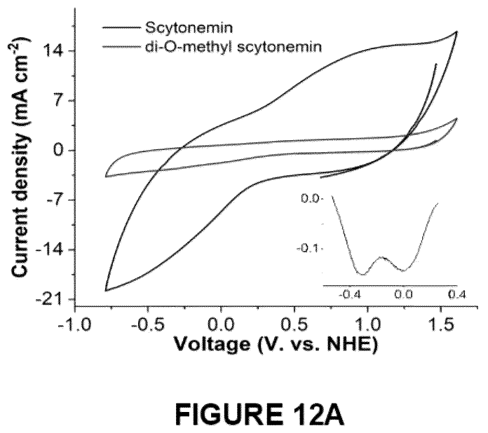

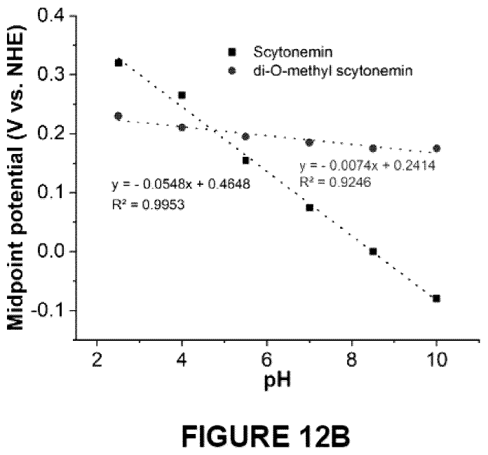

In this example, scytonemin was immobilized on a glassy carbon electrode to evaluate its electrochemical properties in aqueous buffers. Vulcan XC-72 carbon and a proton-conducting Nafion binder were used to facilitate analysis. The CVs of immobilized scytonemin exhibit a reversible redox couple across all pH values studied (pH 2.5-10, FIGS. 4A-4F). A representative CV of scytonemin in pH 8.5 buffer is shown in the black line of FIG. 12A. At all pH values, the Laviron electron transfer rates are faster for the oxidation reaction as is true for the values measured in the acetonitrile electrolyte. The midpoint potential (E_{1/2}) of the scytonemin redox couple shifts to more negative values as the pH increases. In particular aspects, the E_{1/2} values shift by an average of 55 mV per pH unit, which is close to the theoretical value of 59 mV per pH unit as predicted by the Nernst equation. Without being limited to a single theory, it currently is believed that this result is consistent with a reaction mechanism in which the number of protons and electrons transferred at or before the rate-determining step (RDS) is the same (i.e. 2 H+/2 e⁻ for scytonemin).

### Example 6

In this example, analogous CVs of di-O-methyl scytonemin (FIG. 12A, red line and FIGS. 5A-5F), were conducted to evaluate the role of the phenol groups on the electrochemical properties of scytonemin. In this example, the redox peak almost completely disappears upon methylation of the phenol groups. Without being limited to a single theory, it currently is believed that these results suggest that the phenol groups facilitate proton-coupled electron transfer (PCET) to the scytonemin core. Similar findings have been elucidated in biological systems such as cytochrome c oxidase, in which phenol groups on tyrosine residues are essential in facilitating PCET and enabling rapid electron transfer to the enzyme. The CVs of di-O-methyl scytonemin still exhibit reduction and oxidation waves, but their current density is much lower than the phenol-containing scytonemin CVs. The E_{1/2} values of these peaks do not exhibit any significant shift with changing pH (FIG. 12B, line with “⋅” symbol). This result indicates that protons are not transferred at or before the RDS, which suggests that methylating the phenol groups on scytonemin decouples electron transfer from proton transfer. In other words, two successive electron transfer events occur first before subsequent protonation of the anion. Indeed, a close examination of the reductive portion of the CV of di-O-methyl scytonemin reveals two cathodic waves, which are ascribed to the two electron transfer events (FIG. 12A).

### Example 7

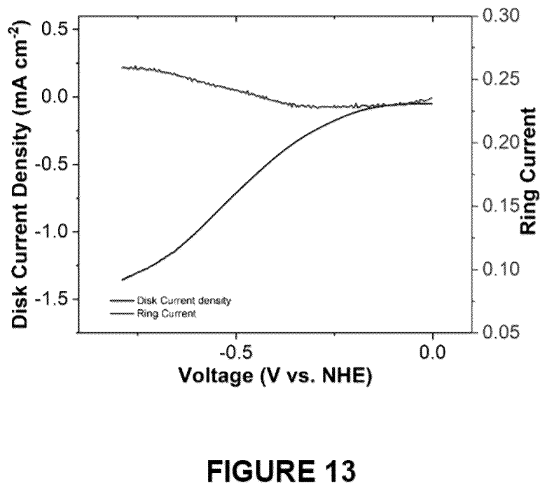

In this example, the ability of scytonemin to quench ROS was evaluated. Rotating ring-disk electrodes (RRDE) O₂ reduction experiments were used to generate in situ ROS on scytonemin-modified electrodes. It is known that glassy carbon electrodes on the disc electrode of RRDE in the absence of scytonemin exclusively produce H₂O₂ via a 2 e⁻/2 H+O₂ reduction process. The Pt ring electrode is held at a positive potential such that the formed H₂O₂ is reoxidized to O₂, and the whole electrode assembly is rotated such that products on the disc are convectively transported to the ring. The amount of H₂O₂ and the average number of electrons by which O₂ is reduced can therefore be calculated by comparing the magnitude of the ring current to that of the disc current. RRDE experiments with scytonemin-modified electrodes indicate that scytonemin quenches in situ generated H₂O₂ at the electrode, resulting in the 4 e⁻/4 H⁺ reduction of O₂ to H₂O (FIG. 13). In particular, the onset potential for O₂ reduction by the scytonemin-modified electrode is about −0.2 V vs. NHE (FIG. 13). Between −0.2 V and −0.3 V, no H₂O₂ current is recorded at the Pt ring (FIG. 13), which indicates that the electrode is selectively reducing O₂ to H₂O via a four-electron pathway. A control experiment with only a carbon electrode without scytonemin shows significant Pt ring current and is known to exclusively reduce O₂ to H₂O₂, which demonstrates that scytonemin effectively quenches ROS at the electrode surface.

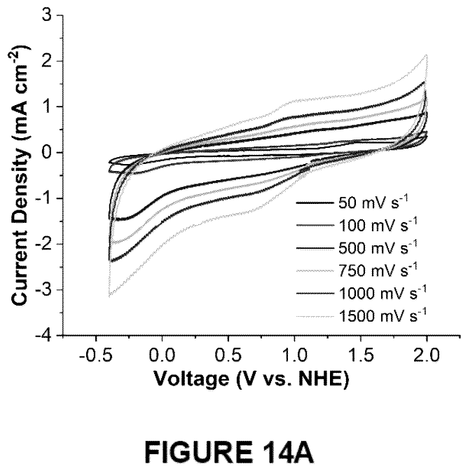

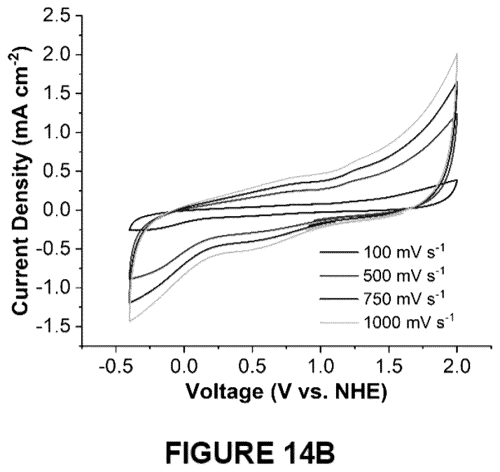

The E_{1/2} value of scytonemin in acetonitrile is about 0.6 V vs. NHE. This redox potential is similar to other well-known antioxidants such as curcumin (FIG. 14A) and daidzein (FIG. 14B), making it plausible that scytonemin functions as an antioxidant in organisms.

Examples according to the disclosure suggest that the redox properties of scytonemin (including analogs and/or derivatives thereof) can turn-over ROS without destroying the UV-screening capabilities. The redox turnover does not readily undergo irreversible reaction with the ROS to form compounds that are not as effective antioxidants nor contain the chromophores to screen UV light as effectively. Additionally, scytonemin (including analogs and/or derivatives thereof) protects from O₂ and ROS diffusion into and out of the cell through the EPS; protection from ROS protection would be particularly important in cases where there is high UV exposure. Also, the reversibility suggests that scytonemin (including analogs and/or derivatives thereof) could deal with a variety of oxidant forms and may be recycled by reduction by an enzyme, exogenous thiol/thiolate, or other exogenous reductants, such as chelated metals (Fe/Cu) found in the exopolysaccharide sheath. Further, oxidation is faster than reduction which is consistent with an ROS/O₂ protection, where the pigment can be recovered by a slower reduction event.

### Example 8

In this example, scytonemin was converted to scytonemin imine (specifically scytonemin hydro-pyrrolo[2,3-b]indole) using aqueous ammonia and acetone. The scytonemin imine structure was confirmed using characterization techniques, focusing on information detailing the structure of the C1-C19 indoline core, which contains the acetone imine motif present in scytonemin imine. HMBC couplings between the H-17 diastereotopic proton resonances (δH 3.71 and 3.24, J=18.9 Hz, δC 54.0 ppm) and the H-19 methyl group (δH 1.91, δC 19.9) to the C-20 imine resonance (δC 173.1) support an acetone imine-derived functionality. Further examination of the HMBC correlations and 13C chemical shift values at the C-3a and C-8b positions support a connectivity of a cyclic imine structure, as shown in below in scytonemin hydro-pyrrolo[2,3-b]indole.

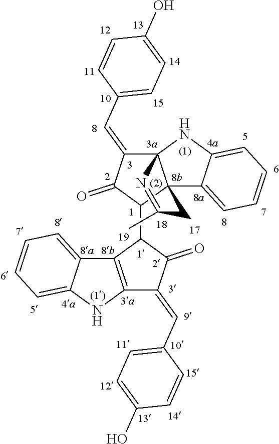

HMBC correlations between the diastereotopic methylene resonances of C-17 and the indoline carbon C-8a (δC 133.6) were used to confirm the structure above, as well as shared correlations between H-5 of the indoline (δH 6.52, δC 109.9) and C-8b (δC 62.2). Additionally, chemical shift values for C-3a (δC 104.8) are more consistent with an N,N-substituted carbon rather than a quaternary nitrogen carbon (δC 63.5). The δC 62.2 resonance is assigned to C-8b based on revised HMBC couplings. HMBC correlations also allowed assignment of exchangeable protons and further support the connectivity of the imine. The exchangeable 1H resonance at 11.9 ppm was assigned to the indole N(1′)-H, supported by HMBC couplings to C3′a (δC 149.2), C8′a (δC 126.4), and C8′b (δC 123.2). The remaining indoline N(1)H proton was assigned to the exchangeable 1H resonance at 6.86 ppm, which exhibited HMBC couplings to the quaternary bridgehead C-3a (δC 104.8) and C-8b (δC 62.2), as well as to the C-4a indole carbon (δC 149.7). Additional ¹H and ¹³C NMR data and assignments are summarized in Table 1.

TABLE 1
¹H and ¹³C NMR data

| | Scytonemin hydro-pyrrolo[2,3-b]indole | | |
|---|---|---|---|
| Position | | δ_{H}[mult., J (Hz), #H] | δ_{C} |

| | | |
|---|---|---|
| 1 | — | n.d. |
| 2 | — | 192.6 |
| 3 | — | n.d. |
| 3a | — | 104.8 |

| | | | |
|---|---|---|---|
| N(1)-H | 6.86 | (s) | — |

| | | |
|---|---|---|
| 4a | — | 149.7 |

| | | | |
|---|---|---|---|
| 5 | 6.52 | (d, 7.8, 1H) | 109.9 |
| 6 | 6.91 | (m, 1H) | 128.2 |
| 7 | 6.5 | (t, 7.8, 1H) | 118.1 |
| 8 | 7.54 | (m, 1H) | 125.4 |

| | | |
|---|---|---|
| 8a | — | 133.6 |
| 8b | — | 62.2 |

| | | | |
|---|---|---|---|
| 9 | 7.4 | (s, 1H) | 135.6 |

| | | |
|---|---|---|
| 10 | — | 124.9 |
| 11 | 8.29 | 135.1 |
| 12 | 6.9 | 115.6 |
| 13 | — | 159.9 |
| 14 | 6.9 | 115.6 |
| 15 | 8.29 | 135.1 |
| 16 (OH) | 10.15 | — |
| 17a | 3.71 | 54.0 |
| 17b | 3.24 | 54.0 |
| 18 | — | 174.8 |
| 19 | 1.95 | 19.4 |
| 20 | — | — |
| 1′ | — | n.d. |
| 2′ | — | 196.2 |
| 3′ | — | n.d. |
| 3′a | — | 149.2 |
| 4′a | — | 141.1 |
| 5′ | 7.54 | 113.1 |
| 6′ | 7.27 | 124.8 |
| 7′ | 7.15 | 121.2 |
| 8′ | 7.24 | 126.6 |
| 8′a | — | 126.4 |
| 8′b | — | 123.2 |
| 9′ | 7.38 | 129.0 |
| 10′ | — | 125.1 |
| 11′ | 7.74 | 131.3 |
| 12′ | 6.97 | 116.3 |
| 13′ | — | 159.6 |
| 14′ | 6.97 | 116.3 |
| 15′ | 7.74 | 131.3 |
| 16′ (OH) | 10.23 | — |

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements unless the context clearly indicates otherwise.

Although the steps of some of the disclosed methods are described in a particular, sequential order for convenient presentation, it should be understood that this manner of description encompasses rearrangement, unless a particular ordering is required by specific language set forth below. For example, steps described sequentially may in some cases be rearranged or performed concurrently. Additionally, the description sometimes uses terms like “produce” or “provide” to describe the disclosed methods. These terms are high-level abstractions of the actual steps that are performed. The actual steps that correspond to these terms will vary depending on the particular implementation and are readily discernible by one of ordinary skill in the art.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing aspects of the disclosure from discussed prior art, the embodiment numbers are not approximates unless the word “about” is recited. Furthermore, not all alternatives recited herein are equivalents.

Certain functional group terms include a symbol “-” which is used to show how the defined functional group attaches to, or within, the compound to which it is bound. Also, a dashed bond (i.e., “---”) as used in certain formulas described herein indicates an optional bond (that is, a bond that may or may not be present). A person of ordinary skill in the art would recognize that the definitions provided below and the compounds and formulas included herein are not intended to include impermissible substitution patterns (e.g., methyl substituted with 5 different groups, and the like). Such impermissible substitution patterns are easily recognized by a person of ordinary skill in the art. In formulas and compounds disclosed herein, a hydrogen atom is present and completes any formal valency requirements (but may not necessarily be illustrated) wherever a functional group or other atom is not illustrated. For example, a phenyl ring that is drawn as

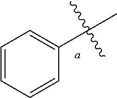 [SMILES: *c1ccccc1]

comprises a hydrogen atom attached to each carbon atom of the phenyl ring other than the “a” carbon, even though such hydrogen atoms are not illustrated. Any functional group disclosed herein and/or defined above can be substituted or unsubstituted, unless otherwise indicated herein.

Compounds disclosed herein may contain one or more asymmetric elements such as stereogenic centers, stereogenic axes and the like, e.g., asymmetric carbon atoms, so that the chemical conjugates can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed by corresponding generic formulas unless context clearly indicates otherwise (e.g., wedge and/or hashed bonds are used) or an express statement excluding an isomer is provided.

In any aspects of the disclosure, any or all hydrogens present in compounds disclosed herein, or in a particular group or moiety within the compound, may be replaced by a deuterium or a tritium. As an example, recitation of “alkyl” includes deuterated and/or tritiated alkyl, where from one to the maximum number of hydrogens present may be replaced by deuterium and/or tritium. For example, methyl refers to both CH₃ or CH₃ wherein from 1 to 3 hydrogens are replaced by deuterium, such as in CDₓH₃₋ₓ.

To facilitate review of the various aspects of the disclosure, the following explanations of specific terms are provided.

Aldehyde: —C(O)H.

Aliphatic: A hydrocarbon group having at least one carbon atom to 50 carbon atoms (C₁-C₅₀), such as one to 25 carbon atoms (C₁-C₂₅), or one to ten carbon atoms (C₁-C₅₀), and which includes alkanes (or alkyl), alkenes (or alkenyl), alkynes (or alkynyl), including cyclic versions thereof, and further including straight- and branched-chain arrangements, and all stereo and position isomers as well. Aliphatic groups may be substituted with one or more groups other than hydrogen, such as aliphatic, heteroaliphatic, haloaliphatic, haloheteroaliphatic, aromatic, an organic functional group, or any combination thereof.

Aliphatic-aromatic: An aromatic group that is or can be coupled to a compound disclosed herein, wherein the aromatic group is or becomes coupled through an aliphatic group. Aliphatic-aromatic groups may be substituted with one or more groups other than hydrogen, such as aliphatic, heteroaliphatic, haloaliphatic, haloheteroaliphatic, aromatic, an organic functional group, or any combination thereof.

Alkenyl: An unsaturated monovalent hydrocarbon having at least two carbon atoms to 50 carbon atoms (C₂-C₅₀), such as two to 25 carbon atoms (C₂-C₂₅), or two to ten carbon atoms (C₂-C₁₀), and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., E or Z). Alkenyl groups may be substituted with one or more groups other than hydrogen, such as aliphatic, heteroaliphatic, haloaliphatic, haloheteroaliphatic, aromatic, an organic functional group, or any combination thereof.

Alkoxy: An exemplary heteroaliphatic group having a formula —O-aliphatic, with examples including, but not limited to, methoxy, ethoxy, n-propoxy, isopropoxy, n-butoxy, t-butoxy, sec-butoxy, n-pentoxy. Alkoxy groups may be substituted with one or more groups other than hydrogen, such as aliphatic, heteroaliphatic, haloaliphatic, haloheteroaliphatic, aromatic, an organic functional group, or any combination thereof.

Alkyl: A saturated monovalent hydrocarbon having at least one carbon atom to 50 carbon atoms (C₁-C₅₀), such as one to 25 carbon atoms (C₁-C₂₅), or one to ten carbon atoms (C₁-C₁₀), wherein the saturated monovalent hydrocarbon can be derived from removing one hydrogen atom from one carbon atom of a parent compound (e.g., alkane). An alkyl group can be branched, straight-chain, or cyclic (e.g., cycloalkyl). Alkyl groups may be substituted with one or more groups other than hydrogen, such as aliphatic, heteroaliphatic, haloaliphatic, haloheteroaliphatic, aromatic, an organic functional group, or any combination thereof.

Alkynyl: An unsaturated monovalent hydrocarbon having at least two carbon atoms to 50 carbon atoms (C₂-C₅₀), such as two to 25 carbon atoms (C₂-C₂₅), or two to ten carbon atoms (C₂-C₁₀), and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl). Alkynyl groups may be substituted with one or more groups other than hydrogen, such as aliphatic, heteroaliphatic, haloaliphatic, haloheteroaliphatic, aromatic, an organic functional group, or any combination thereof.

Amide: —C(O)NR^{a}R^{b} or —NHCOR^{a} wherein each of R^{a} and R^{b} independently is selected from hydrogen, aliphatic, heteroaliphatic, haloaliphatic, aromatic, or any combination thereof. Amide groups may be substituted with one or more groups other than hydrogen, such as aliphatic, heteroaliphatic, haloaliphatic, haloheteroaliphatic, aromatic, an organic functional group, or any combination thereof.

Amine: —NR^{a}R^{b}, wherein each of R^{a} and R^{b} independently is selected from hydrogen, aliphatic, heteroaliphatic, haloaliphatic, aromatic, or any combination thereof. Amine groups may be substituted with one or more groups other than hydrogen, such as aliphatic, heteroaliphatic, haloaliphatic, haloheteroaliphatic, aromatic, an organic functional group, or any combination thereof.

Analog: A compound having a structure according to certain formulas of the present disclosure that comprises one or more different structural components as compared to scytonemin, or a precursor thereto.

Aromatic: A cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Hückel rule (4n+2). The point of attachment to the parent structure typically is through an aromatic portion of the condensed ring system. For example,

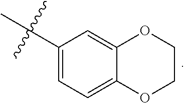 [SMILES: *c1ccc2c(c1)OCCO2]

However, in certain examples, context or express disclosure may indicate that the point of attachment is through a non-aromatic portion of the condensed ring system. For example,

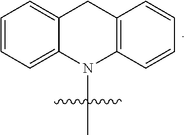 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g., S, O, N, P, or Si), such as in a heteroaryl group or moiety. Aromatic groups may be substituted with one or more groups other than hydrogen, such as aliphatic, heteroaliphatic, haloaliphatic, haloheteroaliphatic, aromatic, an organic functional group, or any combination thereof.

Aryl: An aromatic carbocyclic group comprising at least five carbon atoms to 15 carbon atoms (C₅-C₁₅), such as five to ten carbon atoms (C₅-C₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as aliphatic, heteroaliphatic, haloaliphatic, haloheteroaliphatic, aromatic, an organic functional group, or any combination thereof.

Carboxyl: —C(O)OH or an anion thereof.

Derivative: A compound having a structure according certain formulas of the present disclosure that is created from scytonemin, or a precursor thereto, through one or more chemical reactions.

Disulfide: An exemplary heteroaliphatic group having a formula —SSR^{a}, wherein R^{a} is selected from hydrogen, aliphatic, heteroaliphatic, haloaliphatic, aromatic, or any combination thereof. Disulfide groups may be substituted with one or more groups other than hydrogen, such as aliphatic, heteroaliphatic, haloaliphatic, haloheteroaliphatic, aromatic, an organic functional group, or any combination thereof.

Ester: An exemplary heteroaliphatic group having a formula —C(O)OR^{a} or —OC(O)R^{a}, wherein R^{a} is selected from aliphatic, heteroaliphatic, haloaliphatic, aromatic, or any combination thereof. Ester groups may be substituted with one or more groups other than hydrogen, such as aliphatic, heteroaliphatic, haloaliphatic, haloheteroaliphatic, aromatic, an organic functional group, or any combination thereof.

Haloaliphatic: An aliphatic group wherein one or more hydrogen atoms, such as one to 10 hydrogen atoms, independently is replaced with a halogen atom, such as fluoro, bromo, chloro, or iodo. Haloaliphatic groups may be substituted with one or more groups other than hydrogen, such as aliphatic, heteroaliphatic, haloaliphatic, haloheteroaliphatic, aromatic, an organic functional group, or any combination thereof.

Haloaliphatic-aromatic: An aromatic group that is or can be coupled to a compound disclosed herein, wherein the aromatic group is or becomes coupled through a haloaliphatic group. Haloaliphatic-aromatic groups may be substituted with one or more groups other than hydrogen, such as aliphatic, heteroaliphatic, haloaliphatic, haloheteroaliphatic, aromatic, an organic functional group, or any combination thereof.

Haloalkyl: An alkyl group wherein one or more hydrogen atoms, such as one to 10 hydrogen atoms, independently is replaced with a halogen atom, such as fluoro, bromo, chloro, or iodo. In an independent aspect of the disclosure, haloalkyl can be a CX₃ group, wherein each X independently can be selected from fluoro, bromo, chloro, or iodo. Haloalkyl groups may be substituted with one or more groups other than hydrogen, such as aliphatic, heteroaliphatic, haloaliphatic, haloheteroaliphatic, aromatic, an organic functional group, or any combination thereof.

Haloheteroaliphatic: A heteroaliphatic group comprising at least one halogen atom.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group. Heteroaliphatic groups may be substituted with one or more groups other than hydrogen, such as aliphatic, heteroaliphatic, haloaliphatic, haloheteroaliphatic, aromatic, an organic functional group, or any combination thereof.

Heteroaliphatic-aromatic: An aromatic group that is or can be coupled to a compound disclosed herein, wherein the aromatic group is or becomes coupled through a heteroaliphatic group. Heteroaliphatic-aromatic groups may be substituted with one or more groups other than hydrogen, such as aliphatic, heteroaliphatic, haloaliphatic, haloheteroaliphatic, aromatic, an organic functional group, or any combination thereof.

Heteroalkyl/Heteroalkenyl/Heteroalkynyl: An alkyl, alkenyl, or alkynyl group (which can be branched, straight-chain, or cyclic) comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group. Such groups may be substituted with one or more groups other than hydrogen, such as aliphatic, heteroaliphatic, haloaliphatic, aromatic, haloheteroaliphatic, an organic functional group, or any combination thereof.

Heteroaryl: An aromatic group comprising at least one heteroatom to six heteroatoms, such as one to four heteroatoms, which can be selected from, but not limited to oxygen, nitrogen, sulfur, silicon, boron, selenium, phosphorous, and oxidized forms thereof within the ring. Such heteroaryl groups can have a single ring or multiple condensed rings, wherein the condensed rings may or may not be aromatic and/or contain a heteroatom, provided that the point of attachment is through an atom of the aromatic heteroaryl group. Heteroaryl groups may be substituted with one or more groups other than hydrogen, such as aliphatic, heteroaliphatic, haloaliphatic, aromatic, haloheteroaliphatic, an organic functional group, or any combination thereof.

Heteroatom: An atom other than carbon, such as oxygen, nitrogen, sulfur, silicon, boron, selenium, or phosphorous. In particular aspects of the disclosure, such as when valency constraints do not permit, a heteroatom does not include a halogen atom.

Ketone: —C(O)R^{a}, wherein R^{a} is selected from aliphatic, heteroaliphatic, aromatic, haloaliphatic, or any combination thereof. Ketone groups may be substituted with one or more groups other than hydrogen, such as aliphatic, heteroaliphatic, haloaliphatic, haloheteroaliphatic, aromatic, an organic functional group, or any combination thereof.

Organic Functional Group: A functional group that may be provided by any combination of aliphatic, heteroaliphatic, aromatic, haloaliphatic groups, and/or haloheteroaliphatic, or that may be selected from, but not limited to, aldehyde (i.e., —C(O)H); aroxy (i.e., —O-aromatic); acyl halide (i.e., —C(O)X, wherein X is a halogen, such as Br, F, I, or Cl); halogen; nitro (i.e., —NO₂); cyano (i.e., —CN); azide (i.e., —N₃); carboxyl (i.e., —C(O)OH); carboxylate (i.e., —C(O)O⁻ or salts thereof, wherein the negative charge of the carboxylate group may be balanced with an M⁺ counterion, wherein M⁺ may be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}); amide (i.e., —C(O)NR^{a}R^{b} or —NR^{a}C(O)R^{b} wherein each of R^{a} and R^{b} independently is selected from hydrogen, aliphatic, heteroaliphatic, haloaliphatic, haloheteroaliphatic, or aromatic); ketone (i.e., —C(O)R^{a}, wherein R^{a} is selected from aliphatic, heteroaliphatic, haloaliphatic, haloheteroaliphatic, or aromatic); carbonate (i.e., —OC(O)OR^{a}, wherein R^{a} is selected from aliphatic, heteroaliphatic, haloaliphatic, haloheteroaliphatic, or aromatic); imine (i.e., —C(═NR^{a})R⁶ or —N═CR^{a}R^{b}, wherein R^{a} and R^{b} independently is selected from hydrogen, aliphatic, heteroaliphatic, haloaliphatic, haloheteroaliphatic, or aromatic); azo (i.e., —N═NR^{a} wherein R^{a} is hydrogen, aliphatic, heteroaliphatic, haloaliphatic, haloheteroaliphatic, or aromatic); carbamate (i.e., —OC(O)NR^{a}R^{b}, wherein each of R^{a} and R^{b} independently is selected from hydrogen, aliphatic, heteroaliphatic, haloaliphatic, haloheteroaliphatic, or aromatic); hydroxyl (i.e., —OH); thiol (i.e., —SH); sulfonyl (i.e., —SO₂R^{a}, wherein R^{a} is selected from hydrogen, aliphatic, heteroaliphatic, haloaliphatic, haloheteroaliphatic, or aromatic); sulfonate (i.e., —SO₃⁻, wherein the negative charge of the sulfonate group may be balanced with an M⁺ counter ion, wherein M⁺ may be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}); oxime (i.e., —CR^{a}=NOH, wherein R^{a} is hydrogen, aliphatic, heteroaliphatic, haloaliphatic, haloheteroaliphatic, aromatic, or an organic functional group); sulfonamide (i.e., —SO₂NR^{a}R^{b} or —N(R^{a})SO₂R^{b}, wherein each of R^{a} and R^{b} independently is selected from hydrogen, aliphatic, heteroaliphatic, haloaliphatic, haloheteroaliphatic, aromatic, or an organic functional group); ester (i.e., —C(O)OR^{a} or —OC(O)R^{a}, wherein R^{a} is selected from aliphatic, heteroaliphatic, haloaliphatic, haloheteroaliphatic, aromatic, or an organic functional group); thiocyanate (i.e., —S—CN or —N═C═S); thioketone (i.e., —C(S)R^{a} wherein R^{a} is selected from hydrogen, aliphatic, heteroaliphatic, haloaliphatic, haloheteroaliphatic, aromatic, or an organic functional group); thiocarboxylic acid (i.e., —C(O)SH, or —C(S)OH); thioester (i.e., —C(O)SR^{a} or —C(S)OR^{a} wherein R^{a} is selected from hydrogen, aliphatic, heteroaliphatic, haloaliphatic, haloheteroaliphatic, aromatic, or an organic functional group); dithiocarboxylic acid or ester (i.e., —C(S)SR^{a} wherein R^{a} is selected from hydrogen, aliphatic, heteroaliphatic, haloaliphatic, haloheteroaliphatic, aromatic, or an organic functional group); phosphonate (i.e., —P(O)(OR^{a})₂, wherein each R^{a} independently is hydrogen, aliphatic, heteroaliphatic, haloaliphatic, haloheteroaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}); phosphate (i.e., —O—P(O)(OR^{a})₂, wherein each R^{a} independently is hydrogen, aliphatic, heteroaliphatic, haloaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}); silyl ether (i.e., —OSiR^{a}R^{b}R^{c} wherein each of R^{a}, R^{b}, and R^{c} independently is selected from hydrogen, aliphatic, heteroaliphatic, haloaliphatic, haloheteroaliphatic, aromatic, or an organic functional group); sulfinyl (i.e., —S(O)R^{a}, wherein R^{a} is selected from hydrogen, aliphatic, heteroaliphatic, haloaliphatic, haloheteroaliphatic, aromatic, or an organic functional group); thial (i.e., —C(S)H); or combinations thereof.

Pharmaceutically Acceptable Salt: Pharmaceutically acceptable salts of a compound described herein that are derived from a variety of organic and inorganic counter ions as will be recognizable to those in the art with the benefit of the present disclosure and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. “Pharmaceutically acceptable acid addition salts” are a subset of “pharmaceutically acceptable salts” that retain the biological effectiveness of the free bases while formed by acid partners. In particular, the disclosed compounds can form salts with a variety of pharmaceutically acceptable acids, including, without limitation, inorganic acids such as hydrochloric acid, hydrobromic acid, sulfuric acid, nitric acid, phosphoric acid, and the like, as well as organic acids such as formic acid, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, benzene sulfonic acid, isethionic acid, methanesulfonic acid, ethanesulfonic acid, p-toluenesulfonic acid, salicylic acid, and the like. “Pharmaceutically acceptable base addition salts” are a subset of “pharmaceutically acceptable salts” that are derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.)

Scytonemin: A compound having the structure shown below

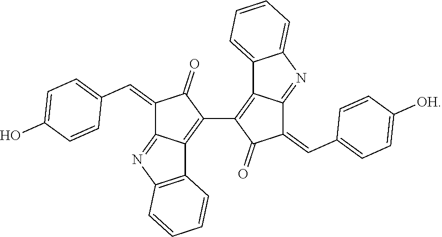 [SMILES: O=C1C(C2=C3C(=Nc4ccccc43)/C(=C\c3ccc(O)cc3)C2=O)=C2C(=Nc3ccccc32)/C1=C\c1ccc(O)cc1]

Scytonemin Imine (or Scytonemin hydro-pyrrolo[2,3-b]indole): A compound having the structure shown below

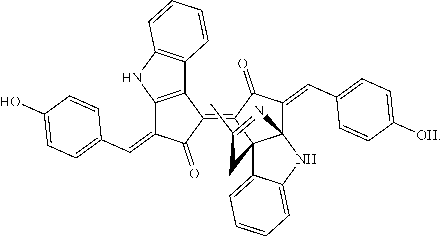

Silyl Ether: An exemplary heteroaliphatic group comprising a silicon atom covalently bound to at least one alkoxy group. Silyl ether groups may be substituted with one or more groups other than hydrogen, such as aliphatic, heteroaliphatic, haloaliphatic, haloheteroaliphatic aromatic, an organic functional group, or any combination thereof.

Subject: Mammals and other animals, such as humans, companion animals (e.g., dogs, cats, rabbits, etc.), utility animals, feed animals and the like; thus, disclosed methods are applicable to both human therapy and veterinary applications.

Therapeutically Effective Amount: An amount of a compound sufficient to protect against reactive oxygen species (“ROS”), provide protection against UV exposure, treat or prevent ROS-induced damage, including intracellular ROS production, and/or to treat a specified disorder or disease related to and/or caused by UV exposure and/or ROS, or to ameliorate or eradicate one or more of its symptoms, and/or to prevent the occurrence of the disease or disorder. The amount of a compound which constitutes a “therapeutically effective amount” will vary depending on the compound, the disease state and its severity, the age of the patient to be treated, and the like. The therapeutically effective amount can be determined by those in the art, particularly with the benefit of the present disclosure.

Treating/Treatment: Treatment of a disease or condition of interest in a subject, particularly a human or mammal having the disease or condition of interest or that may or may not be prone to developing the disease or condition, and includes by way of example, and without limitation: prophylactic administration to prevent the disease or condition from occurring in a subject, or to ameliorate symptoms associated with the condition if required in particular, when such subject is predisposed to the condition but has not yet been diagnosed as having it; inhibiting the disease or condition, for example, arresting or slowing its development; relieving the disease or condition, for example, causing regression of the disease or condition or a symptom thereof; or stabilizing the disease or condition.

As used herein, the terms “disease” and “condition” can be used interchangeably or can be different in that the particular malady or condition may not have a known causative agent (so that etiology has not yet been determined) and it is therefore not yet recognized as a disease but only as an undesirable condition or syndrome, where a more or less specific set of symptoms have been identified by clinicians.

One of ordinary skill in the art will readily appreciate that alternative but functionally equivalent components, materials, designs, and equipment may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skills in the art to employ the present invention.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. It should also be appreciated that the numerical limits may be the values from the examples. Certain lower limits, upper limits and ranges appear in at least one claim below. All numerical values are “about” or “approximately” the indicated value, and consider experimental error and variations that would be expected by a person having ordinary skill in the art.

What is claimed is:

1. A method for making scytonemin, a scytonemin analog, or a scytonemin derivative, wherein the method comprises:
converting a tryptophan-based starting material to an alkene monomer intermediate having a structure according to Formula I;
dimerizing the alkene monomer intermediate by exposing the alkene monomer intermediate to dimerization conditions, wherein
(i) dimerizing the alkene monomer intermediate produces the scytonemin analog; or
(ii) dimerizing the alkene monomer intermediate produces a scytonemin compound precursor having a structure according to Formula II and the method further comprises oxidizing the scytonemin compound precursor to provide scytonemin or the scytonemin derivative;
wherein Formulas I and II are 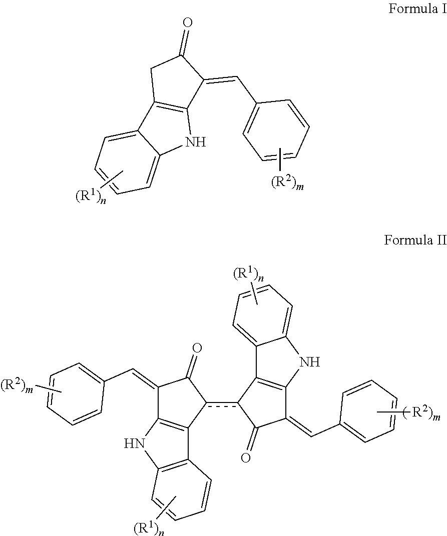
wherein each R¹ independently is selected from aliphatic, heteroaliphatic, haloaliphatic, haloheteroaliphatic, aromatic, or an organic functional group, or two R¹ groups join together to form a fused ring system with carbon atoms to which the two R¹ groups are bound; each R² independently is selected from aliphatic, heteroaliphatic, haloaliphatic, haloheteroaliphatic, aromatic, or an organic functional group, or two R² groups join together to form a fused ring system with carbon atoms to which the two R² groups are bound; n is an integer selected from 0 to 4; and m is an integer selected from 0 to 5.

2. The method for making scytonemin, a scytonemin analog, or a scytonemin derivative of claim 1, wherein oxidizing the scytonemin compound precursor comprises using 2,3-dichloro-5,6-dicyanobenzoquinone.

3. The method for making scytonemin, a scytonemin analog, or a scytonemin derivative of claim 1, wherein the dimerization conditions comprise using an oxidant selected from Cu(OAc)₂, AgF, PhI(OAc)₂, I₂, K₃[Fe(CN)₆], or FeCl₃.

4. The method for making scytonemin, a scytonemin analog, or a scytonemin derivative of claim 1, wherein each R¹ independently is selected from alkyl, alkenyl, alkynyl, heteroalkyl, heteroalkenyl, heteroalkynyl, haloalkyl, haloalkenyl, haloalkynyl, haloheteroalkyl, haloheteroalkenyl, haloheteroalkynyl, aryl, heteroaryl, or an organic functional group selected from OH, SH, NH₂, nitro, or cyano, or wherein two R¹ groups join together to form a 6-membered aromatic ring fused with the phenyl ring to which the two R¹ group are bound.

5. The method for making scytonemin, a scytonemin analog, or a scytonemin derivative of claim 1, wherein each R² independently is selected from alkyl, alkenyl, alkynyl, heteroalkyl, heteroalkenyl, heteroalkynyl, haloalkyl, haloalkenyl, haloalkynyl, haloheteroalkyl, haloheteroalkenyl, haloheteroalkynyl, aryl, heteroaryl, or an organic functional group selected from OH, SH, NH₂, nitro, or cyano.

6. The method for making scytonemin, a scytonemin analog, or a scytonemin derivative of claim 1, wherein the tryptophan-based starting material has a structure according to Formula V

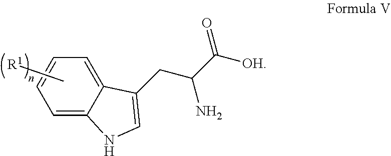

7. The method for making scytonemin, a scytonemin analog, or a scytonemin derivative of claim 1, wherein the scytonemin analog is selected from

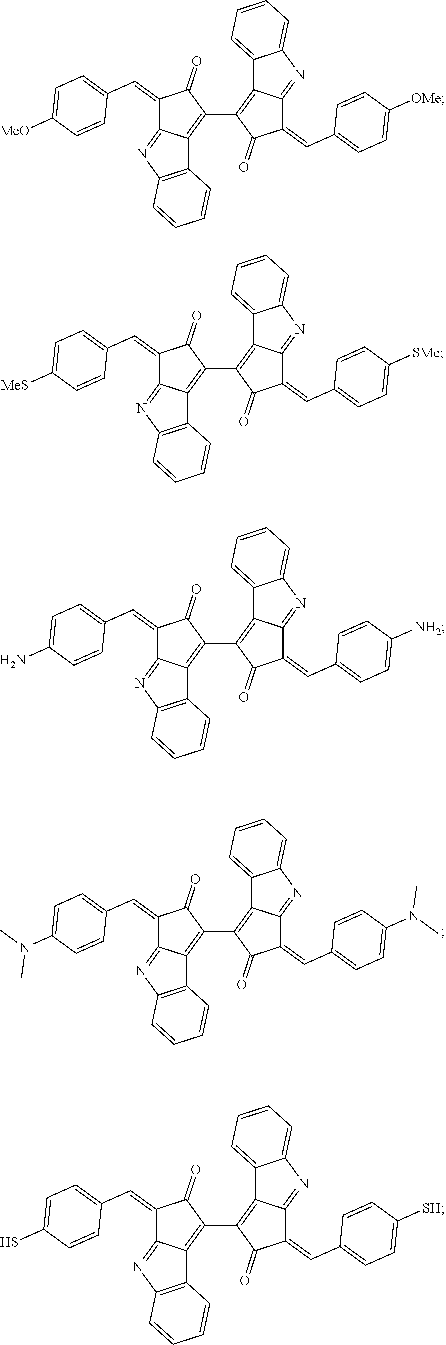

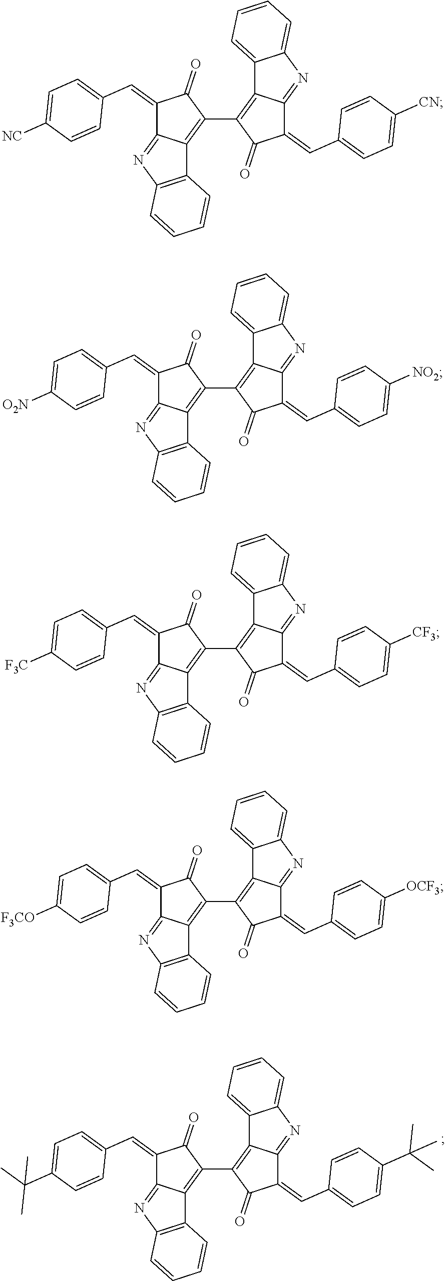 [SMILES: N#Cc1ccc(/C=C2/C(=O)C(C3=C4C(=Nc5ccccc54)/C(=C\c4ccc(C#N)cc4)C3=O)=C3C2=Nc2ccccc23)cc1 ; O=C1C(C2=C3C(=Nc4ccccc43)/C(=C\c3ccc([N+](=O)[O-])cc3)C2=O)=C2C(=Nc3ccccc32)/C1=C\c1ccc([N+](=O)[O-])cc1 ; O=C1C(C2=C3C(=Nc4ccccc43)/C(=C\c3ccc(C(F)(F)F)cc3)C2=O)=C2C(=Nc3ccccc32)/C1=C\c1ccc(C(F)(F)F)cc1 ; O=C1C(C2=C3C(=Nc4ccccc43)/C(=C\c3ccc(OC(F)(F)F)cc3)C2=O)=C2C(=Nc3ccccc32)/C1=C\c1ccc(OC(F)(F)F)cc1 ; CC(C)(C)c1ccc(/C=C2/C(=O)C(C3=C4C(=Nc5ccccc54)/C(=C\c4ccc(C(C)(C)C)cc4)C3=O)=C3C2=Nc2ccccc23)cc1]

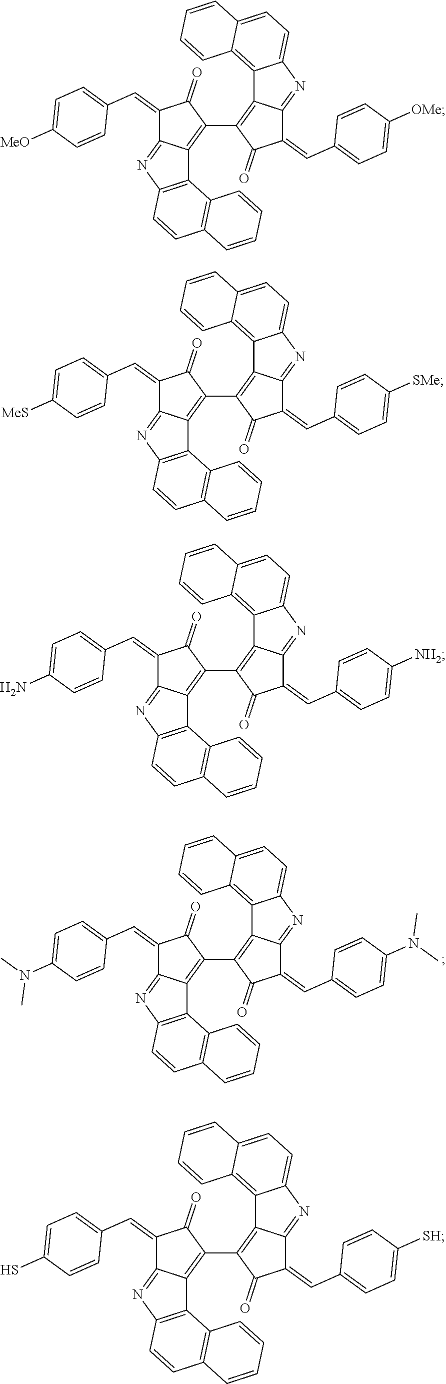

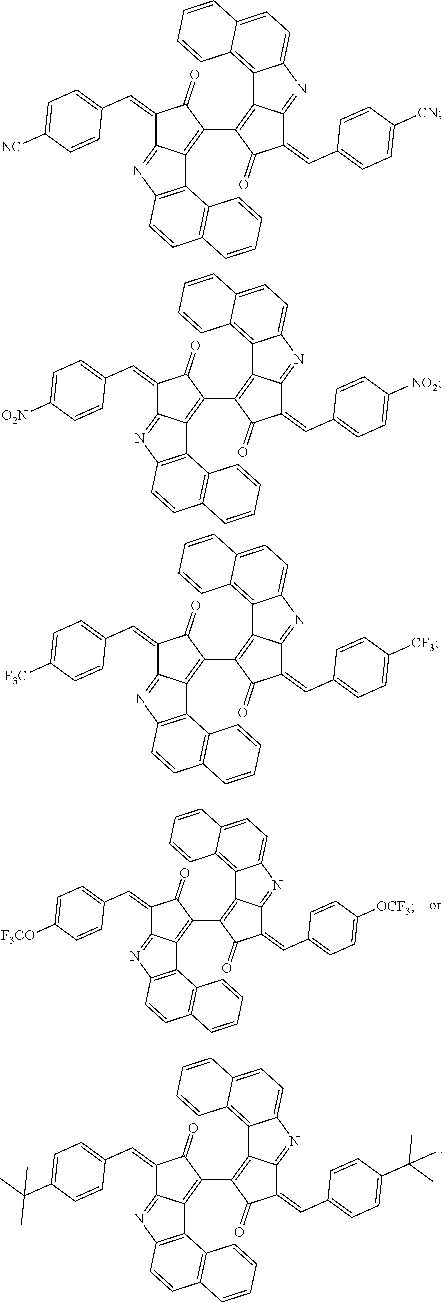

8. The method for making scytonemin, a scytonemin analog, or a scytonemin derivative of claim 1, wherein converting the tryptophan-based starting material to the alkene monomer intermediate comprises:
(i) coupling the tryptophan-based starting material with an anhydride reagent according to Formula III to provide a trihalo oxazolone 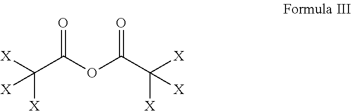
wherein each X independently is a halogen;
(ii) exposing the trihalo oxazolone to a Lewis acid to provide a ketoamide intermediate according to Formula IV 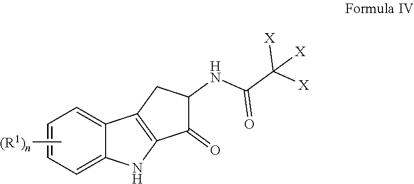
wherein each X independently is a halogen; R¹ is selected from aliphatic, heteroaliphatic, haloaliphatic, haloheteroaliphatic, aromatic, or an organic functional group, or two R¹ groups join together to form a fused ring system with carbon atoms to which the two R¹ groups are bound; and n is an integer selected from 0 to 4;
(iii) oxidizing the ketoamide intermediate to a diketone intermediate;
(iv) performing a Grignard addition reaction to functionalize the diketone intermediate at a position bearing a ketone of the diketone intermediate to provide an alpha-hydroxy ketone-containing compound; and
(v) dehydrating the alpha-hydroxy ketone-containing compound to provide the alkene monomer intermediate.

9. The method for making method for making scytonemin, a scytonemin analog, or a scytonemin derivative of claim 8, wherein the anhydride reagent is selected from

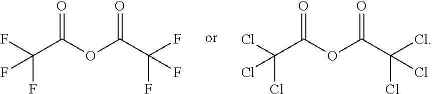 [SMILES: O=C(OC(=O)C(F)(F)F)C(F)(F)F ; "or" ; O=C(OC(=O)C(Cl)(Cl)Cl)C(Cl)(Cl)Cl]

10. The method for making scytonemin, a scytonemin analog, or a scytonemin derivative of claim 8, wherein the trihalo oxazolone is selected from

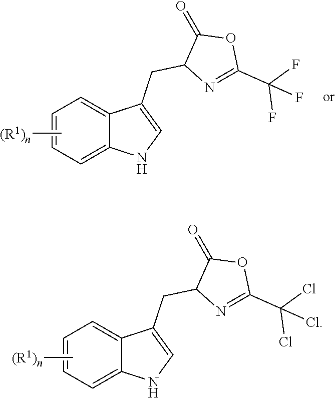

11. The method for making scytonemin, a scytonemin analog, or a scytonemin derivative of claim 8, wherein the ketoamide intermediate is selected from

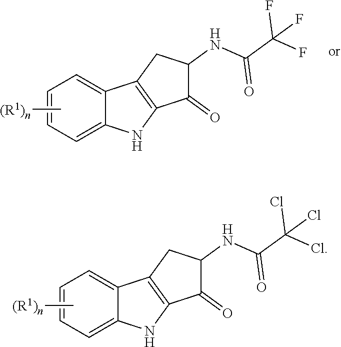

12. The method for making scytonemin, a scytonemin analog, or a scytonemin derivative of claim 8, wherein oxidizing the ketoamide intermediate to the diketone intermediate comprises using CuBr₂, Br₂, N-bromosuccinimide, I₂, PhI(OAc)₂, or tBuOCl.

13. The method for making scytonemin, a scytonemin analog, or a scytonemin derivative of claim 8, wherein the diketone intermediate has a structure according to Formula VI

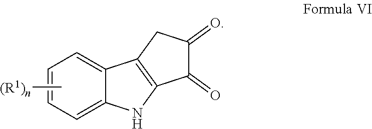

14. The method for making scytonemin, a scytonemin analog, or a scytonemin derivative of claim 8, wherein performing the Grignard addition reaction comprises exposing the diketone intermediate to a reagent having a structure according to a Formula VII 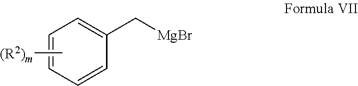
wherein each R² independently is selected from aliphatic, heteroaliphatic, haloaliphatic, haloheteroaliphatic, aromatic, or an organic functional group, or two R² groups join together to form a fused ring system with carbon atoms to which the two R² groups are bound; and m is an integer selected from 0 to 5.

15. The method for making scytonemin, a scytonemin analog, or a scytonemin derivative of claim 8, wherein each R² is selected from OH, OMe, OCF₃, or t-butyl.

16. The method for making scytonemin, a scytonemin analog, or a scytonemin derivative of claim 9, wherein the alpha-hydroxy ketone-containing compound has a structure according to Formula VIII 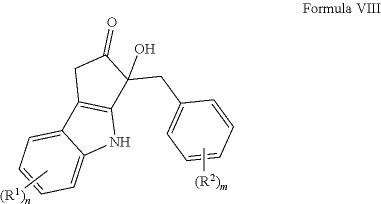
wherein each R¹ independently is selected from aliphatic, heteroaliphatic, haloaliphatic, haloheteroaliphatic, aromatic, or an organic functional group, or two R¹ groups join together to form a fused ring system with carbon atoms to which the two R¹ groups are bound; R² independently is selected from aliphatic, heteroaliphatic, haloaliphatic, haloheteroaliphatic, aromatic, or an organic functional group, or two R² groups join together to form a fused ring system with carbon atoms to which the two R² groups are bound; n is an integer selected from 0 to 4; and m is an integer selected from 0 to 5.

17. The method for making scytonemin, a scytonemin analog, or a scytonemin derivative of claim 8, wherein n is 0 and each m is 1.

18. The method for making scytonemin, a scytonemin analog, or a scytonemin derivative of claim 8, wherein each R² is selected from OH, OMe, OCF₃, or t-butyl.

19. A compound having a formula according to Formula IIA or Formula IIB, 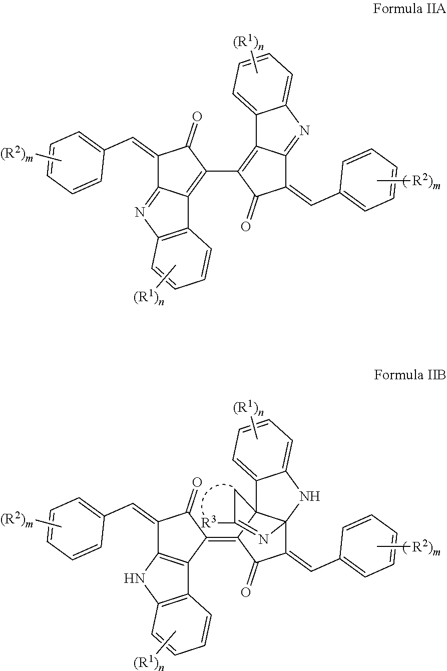
wherein each R¹ independently is selected from aliphatic, heteroaliphatic, haloaliphatic, haloheteroaliphatic, aromatic, or an organic functional group; each R² independently is selected from aliphatic, heteroaliphatic, haloaliphatic, haloheteroaliphatic, aromatic, or an organic functional group; R³ is aliphatic, aromatic, or joins with the carbon atom alpha to the imine carbon atom of Formula IIB to form cyclic group; n is an integer selected from 0 to 4, and m is an integer selected from 0 to 5; provided that the compound is not scytonemin or scytonemin imine.

20. The compound of claim 19, wherein the compound is selected from: 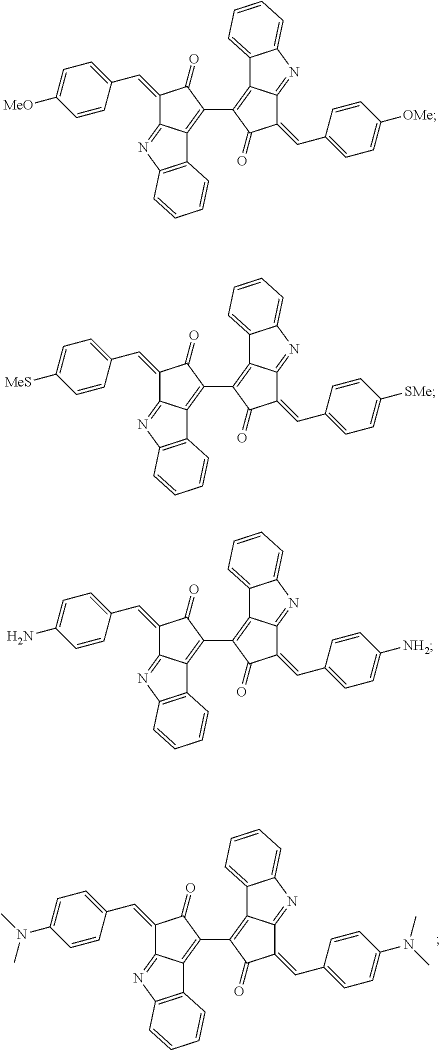 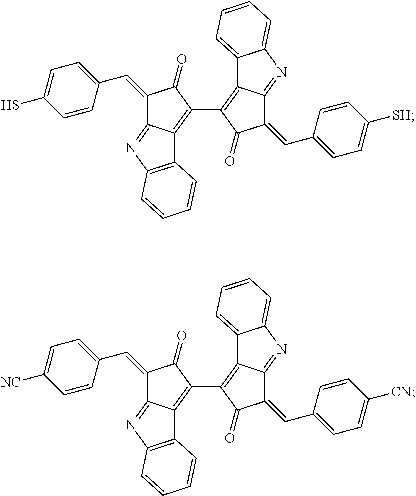 [SMILES: O=C1C(C2=C3C(=Nc4ccccc43)/C(=C\c3ccc(S)cc3)C2=O)=C2C(=Nc3ccccc32)/C1=C\c1ccc(S)cc1 ; N#Cc1ccc(/C=C2/C(=O)C(C3=C4C(=Nc5ccccc54)/C(=C\c4ccc(C#N)cc4)C3=O)=C3C2=Nc2ccccc23)cc1] 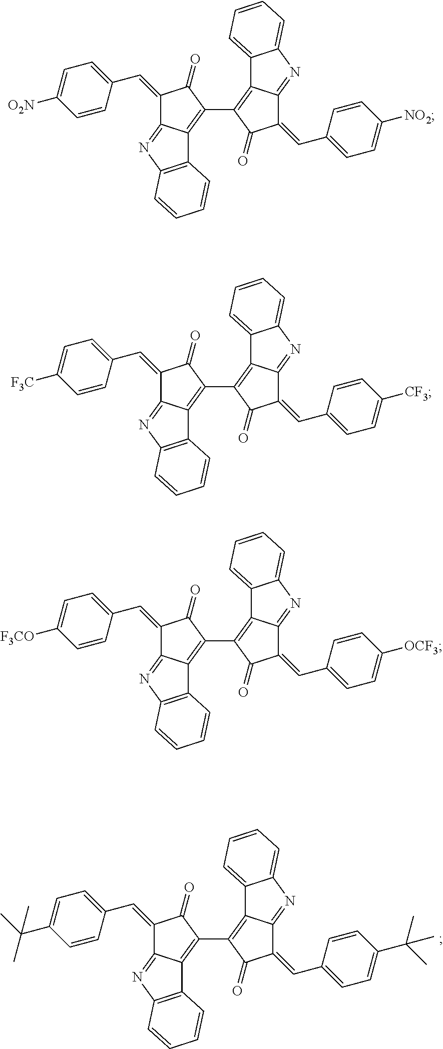 [SMILES: O=C1C(C2=C3C(=Nc4ccccc43)/C(=C\c3ccc([N+](=O)[O-])cc3)C2=O)=C2C(=Nc3ccccc32)/C1=C\c1ccc([N+](=O)[O-])cc1 ; O=C1C(C2=C3C(=Nc4ccccc43)/C(=C\c3ccc(C(F)(F)F)cc3)C2=O)=C2C(=Nc3ccccc32)/C1=C\c1ccc(C(F)(F)F)cc1 ; O=C1C(C2=C3C(=Nc4ccccc43)/C(=C\c3ccc(OC(F)(F)F)cc3)C2=O)=C2C(=Nc3ccccc32)/C1=C\c1ccc(OC(F)(F)F)cc1 ; CC(C)(C)c1ccc(/C=C2/C(=O)C(C3=C4C(=Nc5ccccc54)/C(=C\c4ccc(C(C)(C)C)cc4)C3=O)=C3C2=Nc2ccccc23)cc1] 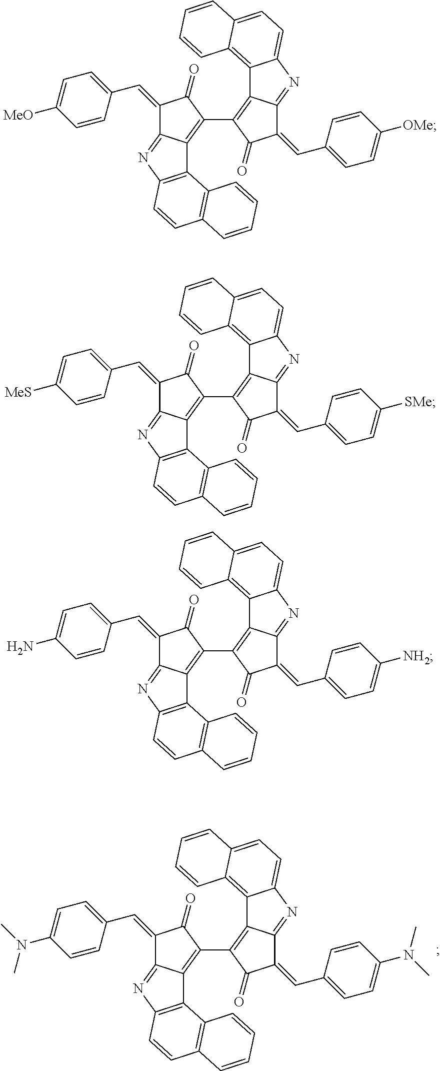 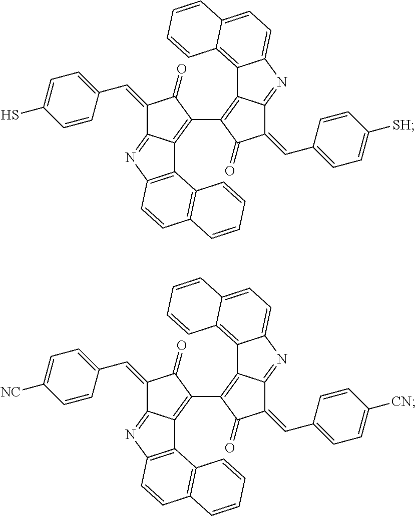 [SMILES: O=C1C(C2=C3C(=Nc4ccc5ccccc5c43)/C(=C\c3ccc(S)cc3)C2=O)=C2C(=Nc3ccc4ccccc4c32)/C1=C\c1ccc(S)cc1 ; N#Cc1ccc(/C=C2/C(=O)C(C3=C4C(=Nc5ccc6ccccc6c54)/C(=C\c4ccc(C#N)cc4)C3=O)=C3C2=Nc2ccc4ccccc4c23)cc1] 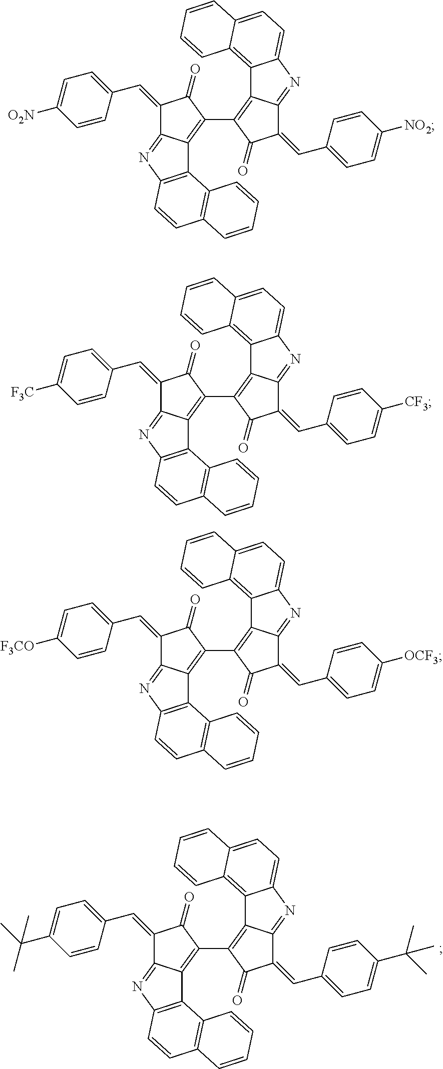 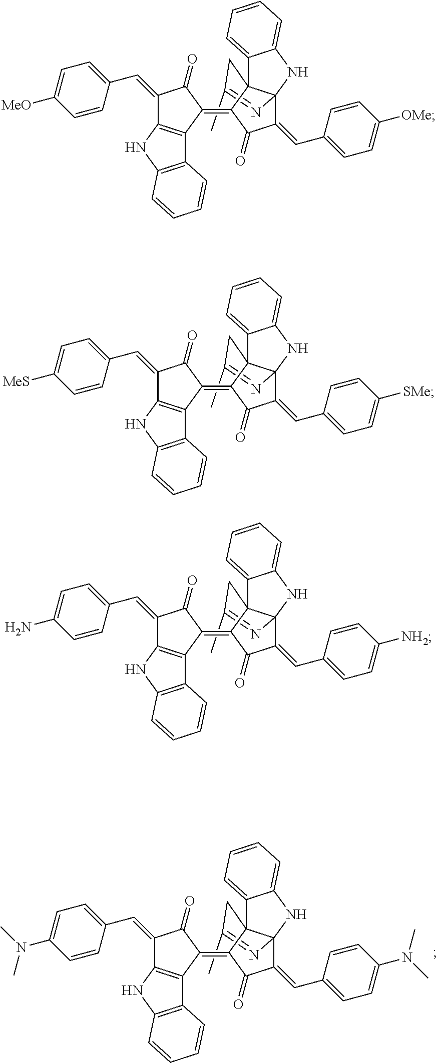 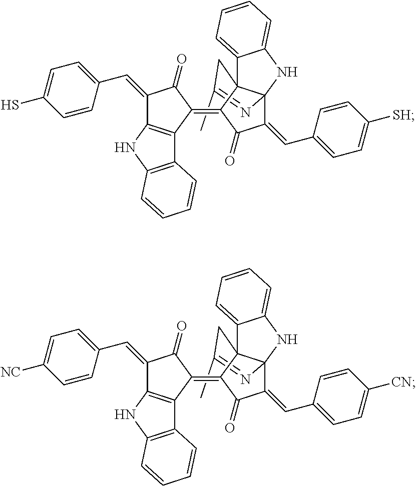 [SMILES: CC1=NC23Nc4ccccc4C2(C1)/C(=C1\C(=O)/C(=C/c2ccc(S)cc2)c2[nH]c4ccccc4c21)C(=O)/C3=C/c1ccc(S)cc1 ; CC1=NC23Nc4ccccc4C2(C1)/C(=C1\C(=O)/C(=C/c2ccc(C#N)cc2)c2[nH]c4ccccc4c21)C(=O)/C3=C/c1ccc(C#N)cc1] 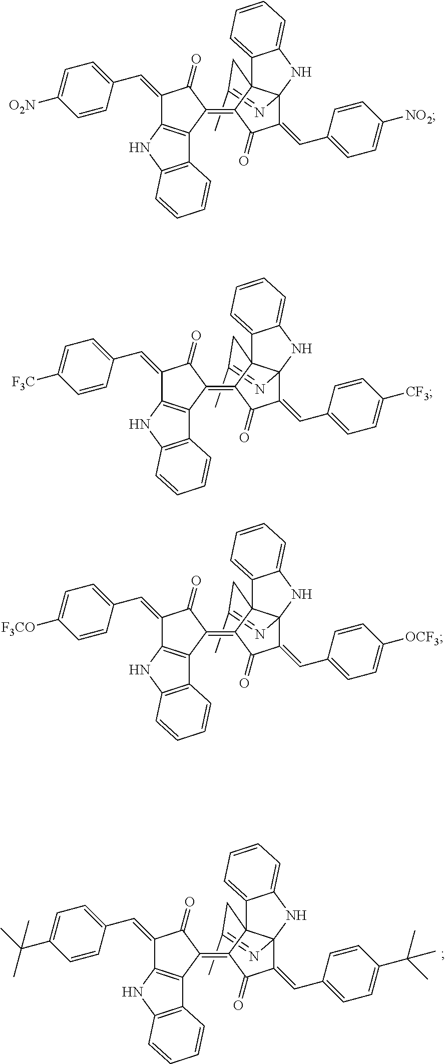 [SMILES: CC1=NC23Nc4ccccc4C2(C1)/C(=C1\C(=O)/C(=C/c2ccc([N+](=O)[O-])cc2)c2[nH]c4ccccc4c21)C(=O)/C3=C/c1ccc([N+](=O)[O-])cc1 ; CC1=NC23Nc4ccccc4C2(C1)/C(=C1\C(=O)/C(=C/c2ccc(C(F)(F)F)cc2)c2[nH]c4ccccc4c21)C(=O)/C3=C/c1ccc(C(F)(F)F)cc1 ; CC1=NC23Nc4ccccc4C2(C1)/C(=C1\C(=O)/C(=C/c2ccc(OC(F)(F)F)cc2)c2[nH]c4ccccc4c21)C(=O)/C3=C/c1ccc(OC(F)(F)F)cc1 ; CC1=NC23Nc4ccccc4C2(C1)/C(=C1\C(=O)/C(=C/c2ccc(C(C)(C)C)cc2)c2[nH]c4ccccc4c21)C(=O)/C3=C/c1ccc(C(C)(C)C)cc1] 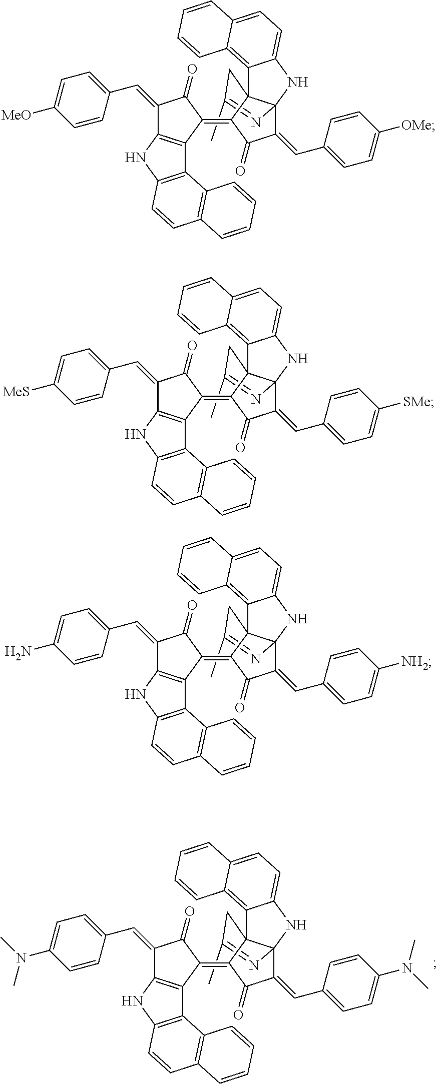 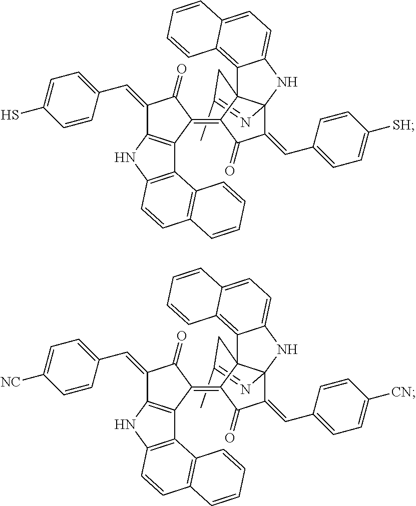 [chemical structure]
